SEQUENCE LISTING (13 sequences)

SEQ ID NO: 1 - protein, 391 aa, synthetic construct
MGGRDIVDGSEFSPSPVPGSQNVPAPAVKKISQYACQRRTTLNNYNQLFTDALDILAENDELRENEGSALAFMRASSVLKSLPFPITSMKDTEGIPSLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYADILESTFEKFKQPSRKVDALDHFQKCFLILKLDHGRVHSEKSGQQEGKGWKAIRVDLVMSPYDRRAFALLGWTGSRQFERDLRRYATHERKMMLDNHALYDRTKRVFLEAESEEEIFAHLGLDYIEPWERNA

SEQ ID NO: 2 - protein, 471 aa, Escherichia coli
MKQSTIALALLPLLFTPVTKARTPEMPVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 3 - protein, 450 aa, Escherichia coli
RTPEMPVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 4 - protein, 449 aa, Escherichia coli
TPEMPVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 5 - protein, 530 aa, Bos taurus
MQGACVLLLLGLWLQLSLAFIPVEEEDPAFWNRQAAQALDVAKKLQPIQKAAKNVILFLGDGMGVPTVTATRILKGQMNDKLGPETPLAMDQFPYVALSKTYNVDRQVPDSAGTATAYLCGVKGNYRTIGVSAAARYNQCNTTRGNEVTSVMNRAKKAGKSVGVVTTTRVQHASPAGAYAHTVNRDWYSDADLPADAQTYGCQDIATQLVNNMDIDVILGGGRKYMFPEGTPDPEYPHDASVNGVRKDKRNLVQEWQAKHQGAQYVWNRTELLQAANDSSVTHLMGLFEPADMKYNVQQDPTKDPTLEEMTEAALQVLSRNPQGFYLFVEGGRIDHGHHDSKAYMALTEAVMFDNAIAKANELTSELDTLILVTADHSHVFSFGGYTLRGTSIFGLAPSKASDKKSYTSILYGNGPGYVLGGGSRPDVNDSISEDPSYRQQAAVPLSSETHGGEDVAVFARGPQAHLVHGVQEETFVAHVMAFAGCVEPYTDCNLPAPSGLSDAAHLAASAPSLALLAGAMLLLLAPALY

SEQ ID NO: 6 - protein, 475 aa, Pandalus borealis
KAYWNKDAQDALDKQLGIKLREKQAKNVIFFLGDGMSLSTVTAARIYKGGLTGKFEREKISWEEFDFAALSKTYNTDKQVTDSAASATAYLTGVKTNQGVIGLDANTVRTNCSYQLDESLFTYSIAHWFQEAGRSTGVVTSTRVTHATPAGTYAHVADRDWENDSDVVHDREDPEICDDIAEQLVFREPGKNFKVIMGGGRRGFFPEEALDIEDGIPGEREDGKHLITDWLDDKASQGATASYVWNRDDLLAVDIRNTDYLMGLFSYTHLDTVLTRDAEMDPTLPEMTKVAIEMLTKDENGFFLLVEGGRIDHMHHANQIRQSLAETLDMEEAVSMALSMTDPEETIILVTADHGHTLTITGYADRNTDILDFAGISDLDDRRYTILDYGSGPGYHITEDGKRYEPTEEDLKDINFRYASAAPKHSVTHDGTDVGIWVNGPFAHLFTGVYEENYIPHALAYAACVGTGRTFCDEK

SEQ ID NO: 7 - protein, 484 aa, Homo sapiens
VIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPACTTD

SEQ ID NO: 8 - protein, 6 aa, synthetic construct
HHHHHH

SEQ ID NO: 9 - DNA, 35 bp, synthetic construct
  source 1..35: mol_type = other DNA
ttttttttttttttttttttttttttttttttttt

SEQ ID NO: 10 - DNA, 60 bp, synthetic construct
  source 1..60: mol_type = other DNA
tttttttttttttttttttttttttttttttttttttttttgcggcgcgtttcgcgccgc

SEQ ID NO: 11 - DNA, 35 bp, synthetic construct
  source 1..35: mol_type = other DNA
ttttttttttttttttttttttttttttttttccc

SEQ ID NO: 12 - DNA, 50 bp, synthetic construct
  source 1..50: mol_type = other DNA
aaccgaccaagctacggttcagaaaattcgcgatgcaattcgcgatcagc

SEQ ID NO: 13 - DNA, 45 bp, synthetic construct
  source 1..45: mol_type = other DNA
ctgacagagatgatgaagtcacatgagacatgaactgagtctttt

(19) United States
(12) Patent Application Publication
(10) Pub. No.: US 2025/0034610 A1
(43) Pub. Date: Jan. 30, 2025

(54) METHODS OF POLYNUCLEOTIDE SYNTHESIS

(71) Applicant: Ansa Biotechnologies, Inc., Emeryville, CA (US)

(72) Inventors: Eric Estrin, Oakland, CA (US); Sebastian Palluk, Oakland, CA (US); Daniel Arlow, San Francisco, CA (US)

(21) Appl. No.: 18/719,073

(22) PCT Filed: Dec. 16, 2022
(86) PCT No.: PCT/US2022/081871

§ 371 Date: Jun. 12, 2024

Related U.S. Application Data
(60) Provisional application No. 63/290,320, filed on Dec. 16, 2021.

(51) Int. Cl. C12P 19/34

(52) U.S. Cl. CPC C12P 19/34

(57) ABSTRACT

Disclosed herein are compositions and methods for de novo enzymatic synthesis of nucleic acid molecules. In particular, and among other things, compositions and methods of the present disclosure provide for reduction of non-terminations in a nucleic acid molecule product generated by de novo enzymatic synthesis. Compositions and methods of the present disclosure also include the use of a phosphatase enzyme to facilitate the reduction of non-terminations.

### CROSS-REFERENCE TO RELATED APPLICATIONS

This application claims priority to and the benefit of U.S. Provisional Application No. 63/290,320, filed on Dec. 16, 2021, the entire disclosure of which is incorporated by reference herein for all purposes.

### FIELD

The present disclosure relates to technologies including methods of nucleic acid synthesis.

### REFERENCE TO A SEQUENCE LISTING

The instant application contains a Sequence Listing (XML file named ABB-006WO_SL.xml, generated on Dec. 13, 2022 and 15,606 bytes in size), which has been submitted electronically and is incorporated by reference herein.

### BACKGROUND

Enzymatic polynucleotide synthesis can be achieved through iterative rounds of template-independent nucleic acid polymerase (e.g. a terminal deoxynucleotidyl transferase) binding to a DNA substrate, incorporation of a nucleotide (e.g. a protected nucleotide) to be added, followed by a deprotection step, allowing for future rounds of nucleotide incorporation. Enzymatic nucleic acid synthesis can result in unwanted additional insertions due to non-termination after addition of a nucleotide. This can result in the addition of more than one nucleotide in a single extension step. Achieving precisely one nucleotide addition in each step is essential for producing accurate synthesis of oligonucleotides, with even small insertion error rates resulting in a high proportion of inaccurate sequences for longer oligonucleotides. Therefore, there is a need for developing improved methods and/or techniques of enzymatic nucleic acid synthesis to prevent processes that lead to more than one nucleotide addition per step.

### SUMMARY

The present disclosure provides, among other things, methods of polynucleotide synthesis. Such methods can be used, for example, to improve accuracy and precision of oligonucleotide synthesis including by improving precision of single nucleotide additions.

As provided herein, in some aspects, are methods of reducing non-terminations in oligonucleotide synthesis reactions.

In some aspects, the present disclosure provides methods comprising contacting conjugate reagents with phosphatases. For instance, in some embodiments, a method comprises: contacting a conjugate reagent comprising a plurality of polymerase-nucleotide conjugates with a phosphatase, wherein said conjugates comprise a nucleotide or modified nucleotide covalently linked to a polymerase via a linker, and wherein said phosphatase is capable of removing one or more terminal 5′ phosphates of an unshielded nucleotide.

In some embodiments, a method of the present disclosure comprises: contacting a conjugate reagent comprising a plurality of polymerase-nucleotide conjugates, wherein said conjugates comprise a nucleotide covalently linked to a polymerase via a linker, and contacting a sample comprising a polynucleotide with said conjugate reagent, wherein said polymerase of the conjugate catalyzes the covalent addition of a nucleotide of the conjugate onto the 3′ hydroxyl of said polynucleotide, and wherein said conjugate reagent is or has been incubated with a phosphatase, wherein said phosphatase is capable of removing a terminal 5′ phosphate of an unshielded nucleotide. In some embodiments, the nucleotide covalently-added is a shielded nucleotide. In some embodiments, the terminal 5′ phosphate can be, for example, an α-phosphate, β-phosphate, χ-phosphate, δ-phosphate, ε-phosphate, ϕ-phosphate, or γ-phosphate of the unshielded nucleotide.

In some embodiments, a method of the present disclosure comprises contacting a said conjugate reagent comprising a polymerase-nucleotide conjugate composition with a phosphatase, wherein said conjugate comprise a nucleotide or modified nucleotide covalently linked to a polymerase via a linker, and wherein said phosphatase is capable of removing a terminal 5′ phosphate of an unshielded nucleotide.

In some embodiments, the present disclosure provides methods of nucleic acid synthesis, comprising: (i) providing a conjugate reagent comprising a plurality of polymerase-nucleotide conjugates, wherein said conjugates each comprise a nucleotide covalently attached to a polymerase via a linker; and (ii) contacting a sample comprising a polynucleotide with said conjugate reagent, wherein said polymerase of the conjugate catalyzes the covalent addition of the nucleotide of the polymerase-nucleotide conjugate onto the 3′ hydroxyl of said polynucleotide, and wherein said conjugate reagent is or has been incubated with a phosphatase, wherein said phosphatase is capable of removing a terminal 5′ phosphate of an unshielded nucleotide. In some embodiments, the covalently-added nucleotide is a shielded nucleotide.

In some embodiments, the present disclosure provides methods comprising: (i) providing a conjugate reagent comprising a polymerase-nucleotide conjugate; and (ii) contacting the conjugate reagent with a phosphatase, wherein said conjugate comprises a nucleotide covalently attached to a polymerase via a linker, and wherein said phosphatase is capable of removing a terminal 5′ phosphate of an unshielded nucleotide.

In some embodiments, the linker is a cleavable linker.

In some embodiments, the present disclosure provides a method of nucleic acid synthesis, comprising: providing a conjugate reagent comprising a plurality of polymerase-nucleotide conjugates, wherein said conjugates comprise a nucleotide or modified nucleotide covalently linked to a polymerase via a linker, and contacting a sample comprising a polynucleotide with said conjugate reagent, wherein said polymerase of the conjugate catalyzes the covalent addition of a shielded nucleotide of the conjugate onto the 3′ hydroxyl of said polynucleotide, and wherein said conjugate reagent is or has been incubated with a phosphatase, wherein said phosphatase is capable of removing one or more terminal 5′ phosphates from an unshielded nucleotide. In some embodiments, the methods provided herein comprise cleaving said linker to remove the polymerase attached to the polynucleotide (e.g., via the linker that tethers the nucleotide to the polymerase) after addition of said conjugate. In some embodiments, the methods provided herein comprise repeating each of the steps of the method one or more times to synthesize a polynucleotide.

In some embodiments, methods provided herein comprise incubating said conjugate reagent with said phosphatase and said incubating hydrolyzes at least one terminal 5′ phosphate of at least one unshielded nucleotide. In some embodiments, methods provided herein comprise incubating said conjugate reagent with said phosphatase. In some embodiments, said incubating of said conjugate reagent with said phosphatase is performed before incubating said sample with said conjugate reagent. In some embodiments, said phosphatase is removed from said conjugate reagent prior to incubating said sample with said conjugate reagent. In some embodiments, said incubating of said conjugate reagent with said phosphatase is performed after contacting said sample with said conjugate reagent.

In some embodiments, the rate of non-termination in said polynucleotide synthesis is decreased as compared to the same synthesis using an otherwise identical conjugate reagent that has not been contacted with a phosphatase.

In some embodiments, said phosphatase does not remove a terminal 5′ phosphate of a shielded nucleotide of said conjugate.

In some embodiments, said unshielded nucleotide is not attached to a polymerase. In some embodiments, said unshielded nucleotide is part of a conjugate: wherein the polymerase is unfolded or improperly folded, wherein the nucleotide is attached to the polymerase such that the nucleotide is not shielded from the phosphatase, or wherein multiple nucleotides are attached to the polymerase. In some embodiments, the phosphatase removes the terminal 5′ phosphate of the unshielded nucleotide.

In some embodiments, said polymerase comprises a template-independent polymerase. In some embodiments, said template-independent polymerase is Terminal deoxynucleotidyl Transferase (TdT), or a variant thereof.

In some embodiments, said phosphatase is an alkaline phosphatase or a non-alkaline phosphatase.

In some embodiments, said polymerase comprises a template-dependent polymerase. In some embodiments, said polymerase comprises a DNA polymerase. In some embodiments, said polymerase comprises an RNA polymerase.

The disclosure provides a composition comprising a plurality of conjugates, wherein each conjugate comprises a nucleotide or modified nucleotide attached to a polymerase, wherein the purity of nucleotides shielded by a linked polymerase of the compositions is greater than about 80%, about 85%, about 90%, about 95%, about 97%, about 98%, about 99%, about 99.5%, or about 99.9%, or about 99.9% with reference to total quantity of nucleotides (shielded and unshielded) in the composition.

In some embodiments, said plurality of conjugates comprises less than about 5%, less than about 4%, less than about 3%, less than about 2%, less than about 1%, less than about 0.5%, less than about 0.1%, less than about 0.05%, or less than about 0.01% of unshielded nucleotides or modified nucleotides.

In some embodiments, compositions provided herein comprise at least one phosphatase. In some embodiments, the phosphatase is an alkaline phosphatase or a non-alkaline phosphatase.

In some embodiments, the plurality of conjugates is capable of extending a nucleic acid molecule by one nucleotide. In some embodiments, the plurality of conjugates is capable of extending a nucleic acid molecule by not more than one nucleotide.

In some embodiments, the nucleic acid molecule is single stranded. In some embodiments, the nucleic acid is double stranded.

In some aspects, the present disclosure provides methods of synthesizing a polynucleotide comprising a pre-determined sequence comprising contacting a nucleic acid molecule with a composition provided herein.

In some embodiments, the method generates a polynucleotide product comprising the pre-determined sequence. In some embodiments, the polynucleotide product has less than about 5%, less than about 4%, less than about 3%, less than about 2%, less than about 1%, less than about 0.5%, less than about 0.1%, less than about 0.05%, or less than about 0.01% of a polynucleotide comprising a sequence that is not the pre-determined sequence as compared to all polynucleotides in the product.

In some embodiments, the nucleic acid molecule is single-stranded. In some embodiments, the nucleic acid molecule is double-stranded. In some embodiments, a method provided herein comprises treating a composition comprising a polymerase-nucleotide conjugate comprising a step of contacting said composition with a phosphatase, wherein said phosphatase is capable of removing a terminal 5′ phosphate of an unshielded nucleotide.

In some embodiments, a method of the present disclosure is method of reducing one or more non-termination reactions in a nucleic acid molecule synthesis, wherein the synthesis is conducted in an environment comprising one or more unshielded nucleotides and wherein the reduction comprises contacting a conjugate reagent comprising a polymerase-nucleotide conjugate with a phosphatase, wherein the polymerase-nucleotide conjugate comprises a nucleotide tethered to the polymerase by a linker, wherein each nucleotide comprises one or more phosphates, and wherein each nucleotide comprises a terminal 5′ phosphate, and wherein the phosphatase is capable of removing the terminal 5′ phosphate from the one or more unshielded nucleotides.

In some embodiments, the number of non-terminations occurring in a synthesis reaction performed in the presence of a phosphatase is reduced as compared to a synthesis conducted under the same conditions but in the absence of a phosphatase.

In some embodiments, the nucleotide is a modified nucleotide.

### BRIEF DESCRIPTION OF THE DRAWINGS

The foregoing and other objects, features and advantages will be apparent from the following description of particular embodiments of the disclosure, as illustrated in the accompanying drawings in which like reference characters refer to the same parts throughout the different views. The drawings are not necessarily to scale, emphasis instead placed upon illustrating the principles of various embodiments of the disclosure.

FIGS. 1A, 1B, and 1C are diagrams of exemplary unshielded nucleotides that could be present in a polymerase-nucleotide conjugate reagent. FIG. 1A shows an exemplary template-independent polymerase with an exemplary unshielded nucleotide (e.g., a deoxynucleoside triphosphate or “dNTP”) tethered in the wrong position.

FIG. 1B shows an exemplary unfolded template-independent polymerase with an exemplary tethered nucleotide (e.g., dNTP).

FIG. 1C illustrates an exemplary “free” (or untethered) nucleotide (e.g., dNTP) as present in an exemplary conjugate reagent. Such free dNTPs can be present in such polymerase-nucleotide conjugate reagent due to, e.g., cleavage of the linker between the nucleotide and the polymerase (e.g., due to instability) or, e.g., due to imperfect removal of free nucleotides from conjugates after conjugate synthesis. Each of the nucleotides in FIG. 1A, FIG. 1B, and FIG. 1C has a 5′ phosphate group accessible to catalytic removal via a phosphatase.

FIG. 1D shows an exemplary polymerase-nucleotide conjugate comprising an exemplary shielded nucleotide (e.g., dNTP). In this figure, the exemplary nucleotide is tethered in the catalytic site of a folded polymerase and is sterically hindered by the tethered polymerase from phosphatase cleavage at its 5′ phosphate.

FIG. 2A shows results of an exemplary single nucleotide addition reaction onto an exemplary single-stranded DNA substrate using an A, C, T, or G polymerase conjugate in the presence (+Phos) or absence (−Phos) of phosphatase. The resulting synthesized oligonucleotides were analyzed by capillary electrophoresis. The x-axes show approximate nucleotide length of oligonucleotides and the y-axes indicate relative fluorescence at 517 nm. Reactions were terminated at the timepoints shown.

FIG. 2B shows an expanded view of the 21 min 41 s timepoint results from FIG. 2A in present or absence of phosphatases. Specific nucleotides are indicated on each set of panels. Arrows designate the +2 additions.

FIG. 3A shows graphical representations of results of capillary electrophoresis analysis of single nucleotide addition reactions onto a single-stranded DNA substrate using a T-polymerase conjugate in the presence of exemplary phosphatase variants from: B. taurus (Quick CIP, NEB), P. borealis (shrimp alkaline phosphatase, NEB), Antarctic bacterium TAB5 (Antarctic phosphatase, NEB), or E. coli (Takara Bio) phosphatase. Synthesis reactions were performed at room temperature (24° C.). A control synthesis reaction was performed without phosphatase. Reactions were terminated at the timepoints shown. The x-axes show relative electrophoretic migration of oligonucleotides (via approximate nucleotide length) and the y-axes indicate relative fluorescence at 517 nm.

FIG. 3B shows graphical representations of results of capillary electrophoresis analysis of an exemplary single nucleotide addition reaction onto a single-stranded DNA substrate using a T-polymerase conjugate in the presence of exemplary phosphatase variants: B. taurus (Quick CIP, NEB), P. borealis (shrimp alkaline phosphatase, NEB), Antarctic bacterium TAB5 (Antarctic phosphatase, NEB), or E. coli (Takara Bio) phosphatase. The synthesis reaction was performed at 37° C. (plus and minus phosphatases) and terminated after 30 minutes. The arrow designates the expected size of +2 additions.

FIG. 4 shows graphical representations of results of an exemplary conjugate-based polynucleotide synthesis of an exemplary 50-mer polynucleotide, conducted in presence or absence of phosphatase, with resulting synthesized polynucleotides distinguished by size along the x-axis using a SeqStudio Genetic Analyzer. Peaks corresponding to the starter oligo and the correct 50-mer synthesis product are labeled.

### DETAILED DESCRIPTION

The details of various embodiments of the disclosure are set forth in the description below. Other features, objects, and advantages of the disclosure will be apparent from the description and the drawings, and from the claims.

### Definitions

As used herein, the term “nucleotide” refers to a molecule comprising a nucleoside and one or more phosphate groups. A “nucleoside” refers to a molecule comprising a nucleobase (e.g. adenine, thymine, cytosine, guanine, or uracil) and a five carbon sugar (e.g. ribose or 2′-deoxyribose). Exemplary nucleotides can be or comprise, without limitation, a nucleoside monophosphate, a nucleoside diphosphate, a nucleoside triphosphate, a nucleoside tetraphosphate, a nucleoside pentaphosphate, or a nucleoside hexaphosphate. As provided herein, TdT and TdT variants, can, in some embodiments, incorporate any nucleoside polyphosphate, including nucleotide analogs comprising modifications to the nucleobase.

As used herein, “non-termination” or “insertion” occurs when more than one nucleotide is added during a single step of a cyclic nucleotide extension. This can occur when an unshielded nucleotide with an uncleaved 5′ phosphate is added to an oligonucleotide.

As used herein, the term “phosphatase” refers to an enzyme capable of removing the 5′ phosphate of a nucleotide, especially a nucleotide that is unshielded as part of an improperly formed conjugate or is not tethered to a polymerase. When not referring to a specific phosphatase enzyme, as used in herein, phosphatase is meant to also include all phosphatase enzymes, engineered enzymes having phosphatase activity, or a functional fragment thereof, that is capable of removing one or more phosphate group(s) from a nucleotide. A phosphatase can also refer to any biomolecule (e.g., a polypeptide or ribozyme) capable of removing one or more phosphate group(s) from a nucleotide, including an engineered enzyme having phosphatase activity, or functional fragments thereof.

As used herein, the term “protected nucleotide” or “shielded nucleotide” refers to a nucleotide that is sterically hindered by a tethered polymerase (or other entity or component such as, e.g., a blocking group) from a phosphatase capable of removing its 5′ phosphate. In some embodiments, such nucleotides are likely to inhibit subsequent nucleotide additions after having been added to an oligonucleotide and before removal of said tethered polymerase.

As used herein, the term “unprotected nucleotide” or “unshielded nucleotide” refers to a nucleotide that is not sterically hindered by a tethered polymerase (or other entity or component such as, e.g., a blocking group) from a phosphatase capable of removing its 5′ phosphate. In some embodiments, an unshielded nucleotide may be tethered to a polymerase, such as in a misfolded polymerase or tethered at an incorrect position. An unshielded nucleotide may be untethered (or free) from a polymerase. Unshielded nucleotides that have not been exposed to phosphatase are more likely to be erroneously added to a polynucleotide as an insertion after a shielded nucleotide has been properly added.

### Polymerase-Nucleotide Conjugates & Polynucleotide Synthesis

Among other things, the present disclosure provides polymerase nucleotide conjugates. As is understood to those in the art, there are various challenges associated with precise and accurate polynucleotide synthesis. For example, among other things, polymerases can erroneously catalyze covalent addition of nucleotides, which may result in the addition of more than one nucleotide per step when using polymerase-nucleotide conjugates in a controlled, step-wise nucleic acid synthesis (e.g., insertion or non-termination). Technologies provided herein, including combining such conjugates with phosphatases (e.g., providing a polymerase-nucleotide conjugate in the presence of a phosphatase) overcome such challenges. These technologies help to achieve more accurate and precise stepwise additions, reducing errors as compared to previously described synthesis approaches (e.g., those conducted in the absence of phosphatases).

In some embodiments, the conjugates are provided in the presence of a phosphatase. In some embodiments, the present disclosure provides a conjugate reagent comprising a plurality of polymerase-nucleotide conjugates, wherein a polymerase and a nucleotide are linked via a linker. In some embodiments, the linker is cleavable.

In some embodiments, the conjugate reagent exists in the presence of phosphatases.

In some embodiments, the polymerase-nucleotide conjugates are combined with a template (e.g., a start oligo or initial oligonucleotide) in the presence of a phosphatase.

A typical process for stepwise synthesis of a polynucleotide comprises adding individual nucleotides step-wise to a starter oligo (i.e., an initial oligonucleotide) via cyclical steps. For example, in some embodiments, the steps comprise: addition of a polymerase-nucleotide conjugate to an oligonucleotide, covalent addition of the nucleotide to the 3′ end of the oligonucleotide catalyzed by the polymerase, and cleavage of the polymerase from the added nucleotide. These steps can be repeated until a desired elongated polynucleotide is synthesized such that the elongated polynucleotide has a length one or more nucleotides longer than the polynucleotide prior to the steps being repeated one or more times.

Among other things, provided herein are methods of nucleic acid synthesis. In some embodiments, a method of nucleic acid synthesis comprises a step of contacting (e.g., incubating) a conjugate reagent comprising polymerase-nucleotide conjugates (e.g., a plurality of polymerase-nucleotide conjugates) in the presence of one or more phosphatases. In some embodiments, the nucleotides in the plurality are the same nucleotides (e.g., A, G, T, or C, etc.). In some embodiments, the nucleotides are different nucleotides (e.g., A, G, T, and/or C, etc.) In some such embodiments, synthesis conducted in the presence of a phosphatase is improved in one or more ways (e.g., more precise, more efficient, more accurate) as compared to the same synthesis in the absence of a phosphatase.

In some embodiments, the synthesis performed in the presence of a phosphatase prevents addition of unshielded nucleotides to a nucleic acid. The methods provided herein comprise a step of contacting (e.g., incubating) a conjugate reagent comprising a plurality of polymerase-nucleotide conjugates with a phosphatase, wherein there is a reduction in rates of processes that lead to addition of more than one nucleotide per step when using polymerase-nucleotide conjugates in nucleic acid synthesis (e.g. non-termination leading to an additional nucleotide insertion) as compared to synthesis without phosphatase or without treatment of the conjugate reagent with phosphatase.

### Non-Termination

Achieving precisely one nucleotide addition in each step of a stepwise nucleic acid synthesis is essential for producing accurate synthesis of longer oligonucleotides. It remains a challenge in the industry to conduct these additions with precision and accuracy. For example, stepwise nucleic acid synthesis using polymerase-nucleotide conjugates may be susceptible to insertions and/or non-termination resulting in the addition of more than one nucleotide to a nucleic acid in a single step of a cyclic nucleotide extension.

An unshielded nucleotide is not sterically-hindered or is only partially sterically-hindered by a tethered polymerase from phosphatase cleavage at its 5′ phosphate. During oligonucleotide synthesis, a polymerase can erroneously catalyze the covalent addition of the unshielded nucleotide, which may result in the addition of more than one nucleotide per step when using polymerase-nucleotide conjugates in nucleic acid synthesis (e.g., insertion or non-termination). Technologies provided herein help overcome this challenge to achieve accurate and precise stepwise addition with reduced errors as compared to previously described synthesis approaches.

In some embodiments, non-termination may occur when an unshielded nucleotide with an uncleaved 5′ phosphate is added to an oligonucleotide. As provided herein, in some embodiments, a phosphatase hydrolyzes a 5′ phosphate (e.g. a terminal 5′ phosphate) of a nucleotide (e.g., of a nucleotide triphosphate, etc.). In some embodiments, the terminal 5′ phosphate is on an α-phosphate, β-phosphate, χ-phosphate, δ-phosphate, ε-phosphate, ϕ-phosphate, or γ-phosphate of the nucleotide. In some embodiments, a phosphatase as disclosed herein hydrolyzes a 5′ phosphate (e.g., a terminal 5′ phosphate) of a nucleotide in a polymerase-nucleotide conjugate or of a free nucleotide. In some embodiments, a phosphatase as disclosed herein hydrolyzes 5′ phosphates of nucleotides, e.g., one or more 5′ terminal phosphate(s) of nucleotides in a plurality of polymerase-nucleotide conjugates, and prevents the hydrolyzed nucleotide from addition to the nucleic acid during oligonucleotide synthesis. In some embodiments, a phosphatase hydrolyzes a 5′ phosphate of an unshielded nucleotide of a polymerase-nucleotide conjugate. In some such embodiments, the hydrolysis of the 5′ phosphate prevents the unshielded nucleotide from addition to the nucleic acid during oligonucleotide synthesis. In some embodiments, a phosphatase as disclosed herein hydrolyzes one or more 5′ phosphate(s) of unshielded nucleotides in a plurality of polymerase-nucleotide conjugates and prevents said unshielded nucleotides from addition to the nucleic acid during oligonucleotide synthesis. In some embodiments, a phosphatase as disclosed herein hydrolyzes one or more 5′ phosphate(s) of one or more free nucleotides in a composition comprising one or more polymerase-nucleotide conjugates and prevents the free nucleotides from addition to the nucleic acid during oligonucleotide synthesis. In some embodiments, a phosphatase hydrolyzes a 5′ phosphate of one or more free nucleotides present in a composition comprising one or more polymerase-nucleotide conjugates. In some such embodiments, the hydrolysis of the 5′ phosphate prevents the one or more free nucleotides from addition to the nucleic acid during oligonucleotide synthesis.

### Unshielded Nucleotides

The presence of an unshielded nucleotide in a conjugate reagent can lead to non-termination (i.e. insertion) in oligonucleotide synthesis. In some embodiments, an unshielded nucleotide is less likely to inhibit subsequent nucleotide addition after having been added to an oligonucleotide during nucleic acid synthesis.

In some embodiments, a fraction of nucleotides in a plurality of polymerase-nucleotide conjugates are not shielded by a polymerase. In some embodiments, a polymerase-nucleotide conjugate comprises an unshielded nucleotide. In some embodiments, a polymerase molecule in a polymerase-nucleotide conjugate does not sterically hinder access of a phosphatase to the 5′ phosphate of a tethered nucleotide. In some embodiments, a tethered nucleotide is an unshielded nucleotide. In some embodiments, an unshielded nucleotide in a polymerase-nucleotide conjugate is not sterically hindered by a tethered polymerase from a phosphatase capable of removing its 5′ phosphate. In some embodiments, removing the 5′ phosphate (e.g., terminal 5′ phosphate) of a nucleotide in a polymerase-nucleotide conjugate prevents the nucleotide from addition to the nucleic acid during nucleic acid synthesis. In some embodiments, a phosphatase hydrolyzes the 5′ phosphate (e.g., terminal 5′ phosphate) of an unshielded nucleotide in a polymerase-nucleotide conjugate. In some such embodiments, more than one 5′ terminal phosphate is removed, for example, wherein a 5′ terminal phosphates is removed serially, i.e., from a first nucleotide, then a second nucleotide, etc. Thus, in some such embodiments, one or more 5′ terminal phosphates may be removed, though in a given nucleotide, a single 5′ terminal phosphate exists and is removed, upon which point a different phosphate becomes the 5′ terminal phosphate of a nucleotide having at least one 5′ terminal phosphate.

In some embodiments, an unshielded nucleotide is part of an improperly formed conjugate. In some embodiments, an improperly formed conjugate comprises a mis-folded polymerase, a polymerase in which a nucleotide is attached in the wrong position, and/or a polymerase in which multiple nucleotides are attached. In some embodiments, a nucleotide is free or untethered from a polymerase due to instability or imperfect purification. In some embodiments, a free or untethered nucleotide is an unshielded nucleotide.

### Examples of Unshielded Nucleotides

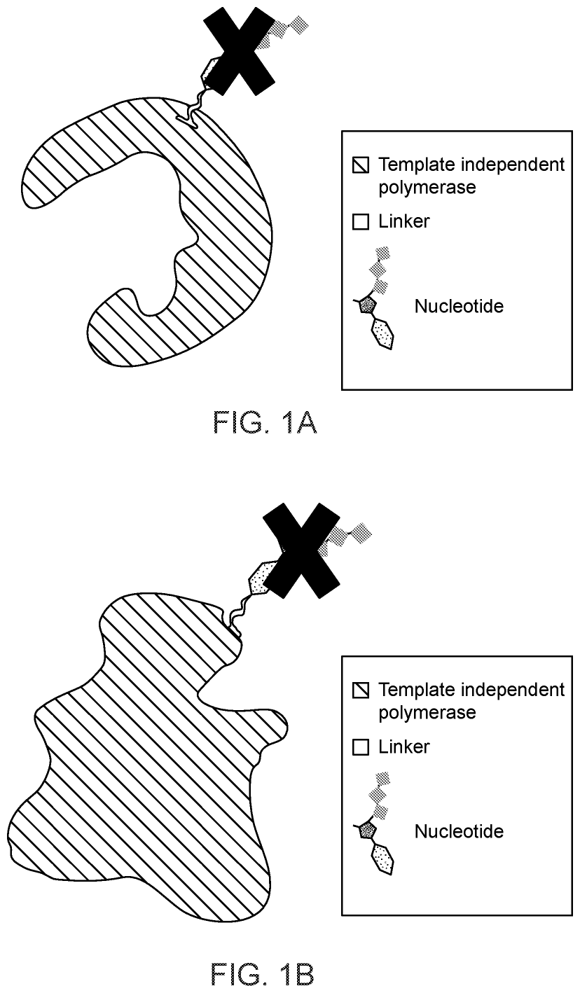

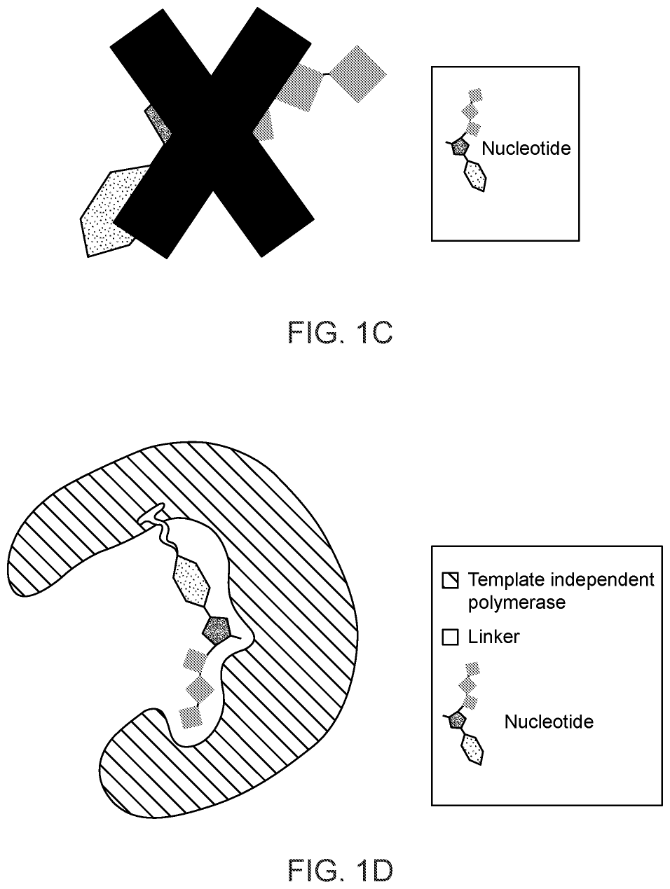

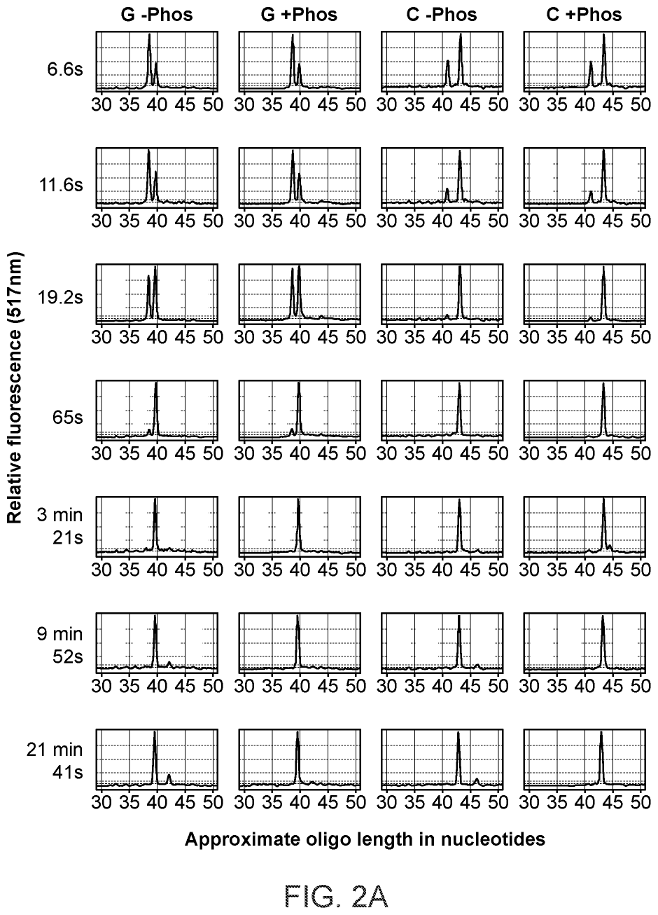

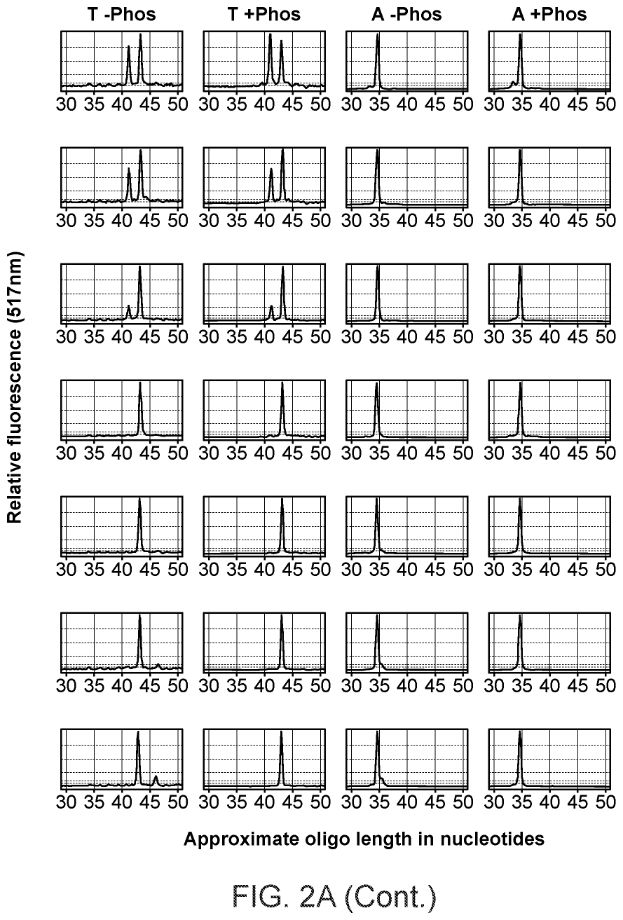

Lack of shielding of a nucleotide in a polymerase-nucleotide conjugate can occur due to a number of processes during preparation of polymerase-nucleotide conjugates or during the addition reaction itself. Nucleotides that are not attached to a polymerase in a composition comprising a polymerase-nucleotide conjugate are considered unshielded nucleotides. Non-limiting examples of processes that may result in a polymerase-nucleotide conjugate comprising an unshielded nucleotide include: spontaneous cleavage of a linker between a nucleotide and a polymerase (see, e.g., linkers in FIGS. 1A-1D), unfolding of a polymerase (see, e.g., FIG. 1B), a polymerase having a nucleotide attached in the wrong position (see, e.g., FIG. 1A), a polymerase comprising multiple attached nucleotides (i.e., on a single polymerase), or an untethered, free nucleotide (see, e.g., FIG. 1C). In some embodiments, spontaneous cleavage of a linker between a nucleotide and a polymerase may occur due to instability, resulting in free nucleotides in the conjugate reagent.

### Shielded Nucleotide

In some embodiments, a fraction of nucleotides in a plurality of polymerase-nucleotide conjugates are shielded by a polymerase. A non-limiting example of a shielded nucleotide includes a nucleotide that is tethered in the catalytic site of a correctly folded polymerase (see, e.g., exemplary schematic in FIG. 1D). In some embodiments, a polymerase-nucleotide conjugate comprises a shielded nucleotide. In some embodiments, a polymerase molecule in a polymerase-nucleotide conjugate sterically hinders access of a phosphatase to the 5′ phosphate (e.g., the 5′ terminal phosphate) of a tethered nucleotide. In some embodiments, the 5′ phosphate (e.g., the 5′ terminal phosphate) can be, for example, on an α-phosphate, β-phosphate, χ-phosphate, δ-phosphate, ε-phosphate, ϕ-phosphate, or γ-phosphate. In some embodiments, a tethered nucleotide is a shielded nucleotide. In some embodiments, a shielded nucleotide in a polymerase-nucleotide conjugate is sterically hindered by a tethered polymerase from a phosphatase capable of removing its 5′ phosphate. In some embodiments, a phosphatase is unable to hydrolyze the 5′ phosphate (e.g., 5′ terminal phosphate) of a shielded nucleotide in a polymerase-nucleotide conjugate.

### Phosphatase

A phosphatase typically uses water to cleave a phosphoric acid monoester into a phosphate ion and an alcohol. A phosphatase enzyme catalyzes the hydrolysis of its substrate.

The κ′ phosphate of a nucleotide in a polymerase-nucleotide conjugate is necessary for addition of the nucleotide to an oligonucleotide by a polymerase. In some embodiments, removal of the 5′ phosphate of a nucleotide in a polymerase-nucleotide conjugate prevents the nucleotide from addition to the nucleic acid during oligonucleotide synthesis.

Disclosed herein are methods comprising adding a phosphatase to a conjugate reagent to hydrolyze the 5′ phosphate group of a nucleotide in a polymerase-nucleotide conjugate. In some embodiments, a phosphatase removes a phosphate moiety from an unshielded nucleotide in a polymerase-nucleotide conjugate. In some embodiments, the methods comprise adding a phosphatase capable of hydrolyzing a 5′ phosphate group of an unshielded nucleotide to a polymerase-nucleotide conjugate.

### Types of Phosphatases

Any suitable phosphatase, engineered enzyme having phosphatase activity, or a functional fragment thereof for the methods described herein is contemplated by the disclosure. In some embodiments, the phosphatase is a nucleotidase. Enzymes having phosphatase activity are included in the enzyme class E.C 3.1.3 . . . , hydrolases acting on ester bonds, e.g. a phosphoric monoester hydrolase. However, enzymes having suitable phosphatase activity (such as apyrase) may be found in other enzyme classes. In some embodiments, a phosphatase may optionally be capable of hydrolyzing an inorganic phosphate substrate, e.g. pyrophosphate.

In some embodiments, the phosphatase is immobilized to a solid support. In some embodiments, the phosphatase is a fusion protein. In some embodiments, the phosphatase comprises a detectable label. In some embodiments, the phosphatase is a recombinant polypeptide. In some embodiments, the phosphatase is a wild type phosphatase. In some embodiments, the wild type phosphatase is isolated from the organism in which it is natively expressed.

Alkaline phosphatases (ALP, ALKP, ALPase, Alk Phos), or basic phosphatases, are plasma membrane-bound glycoproteins that catalyze the hydrolysis of phosphate monoesters and are optimally active at alkaline pH environments. Alkaline phosphatases are homodimeric protein enzymes of 86 kilodaltons. Each monomer contains five cysteine residues, two zinc atoms, and one magnesium atom crucial to its catalytic function.

Non-limiting examples of alkaline phosphatases include: B. taurus (Quick calf-intestinal alkaline phosphatase, or CIP, NEB), Pandalus borealis (shrimp alkaline phosphatase, NEB), Antarctic bacterium TAB5 (Antarctic phosphatase, NEB), and E. coli (Takara Bio) phosphatase. Additional non-limiting examples of alkaline phosphatases include: placental alkaline phosphatase (PLAP) and human-intestinal alkaline phosphatase.

Non-alkaline phosphatases may be acid phosphatases. A non-limiting example of a non-alkaline phosphatases is tartrate resistant acid phosphatase.

Illustrative amino acid sequences encoding phosphatases for use in the methods described herein are shown, without limitation, in Table 1.

TABLE 1
Exemplary Alkaline Phosphatase Sequences

| Phosphatase | Amino Acid Sequence | SEQ ID NO |
|---|---|---|
| Alkaline | MKQSTIALALLPLLFTPVTKARTPEMPVLENRAAQGDITAPGGARRL | 2 |
| Phosphatase | TGDQTAALRDSLSDKPAKNIILLIGDGMGDSEITAARNYAEGAGGFF | |
| (E. coli) | KGIDALPLTGQYTHYALNKKTGKPDYVTDSAASATAWSTGVKTYNGA | |
| | LGVDIHEKDHPTILEMAKAAGLATGNVSTAELQDATPAALVAHVTSR | |
| | KCYGPSATSEKCPGNALEKGGKGSITEQLLNARADVTLGGGAKTFAE | |
| | TATAGEWQGKTLREQAQARGYQLVSDAASLNSVTEANQQKPLLGLFA | |
| | DGNMPVRWLGPKATYHGNIDKPAVTCTPNPQRNDSVPTLAQMTDKAI | |
| | ELLSKNEKGFFLQVEGASIDKQDHAANPCGQIGETVDLDEAVQRALE | |
| | FAKKEGNTLVIVTADHAHASQIVAPDTKAPGLTQALNTKDGAVMVMS | |
| | YGNSEEDSQEHTGSQLRIAAYGPHAANVVGLTDQTDLFYTMKAALGL | |
| | K | |
| Alkaline | RTPEMPVLENRAAQGDITAPGGARRLTGDQTAALRDSLSDKPAKNII | 3 |
| Phosphatase- | LLIGDGMGDSEITAARNYAEGAGGFFKGIDALPLTGQYTHYALNKKT | |
| Isozyme 1 | GKPDYVTDSAASATAWSTGVKTYNGALGVDIHEKDHPTILEMAKAAG | |
| (E. coli) | LATGNVSTAELQDATPAALVAHVTSRKCYGPSATSEKCPGNALEKGG | |
| | KGSITEQLLNARADVTLGGGAKTFAETATAGEWQGKTLREQAQARGY | |
| | QLVSDAASLNSVTEANQQKPLLGLFADGNMPVRWLGPKATYHGNIDK | |
| | PAVTCTPNPQRNDSVPTLAQMTDKAIELLSKNEKGFFLQVEGASIDK | |
| | QDHAANPCGQIGETVDLDEAVQRALEFAKKEGNTLVIVTADHAHASQ | |
| | IVAPDTKAPGLTQALNTKDGAVMVMSYGNSEEDSQEHTGSQLRIAAY | |
| | GPHAANVVGLTDQTDLFYTMKAALGLK | |
| Alkaline | TPEMPVLENRAAQGDITAPGGARRLTGDQTAALRDSLSDKPAKNIIL | 4 |
| Phosphatase- | LIGDGMGDSEITAARNYAEGAGGFFKGIDALPLTGQYTHYALNKKTG | |
| Isozyme 2 | KPDYVTDSAASATAWSTGVKTYNGALGVDIHEKDHPTILEMAKAAGL | |
| (E. coli) | ATGNVSTAELQDATPAALVAHVTSRKCYGPSATSEKCPGNALEKGGK | |
| | GSITEQLLNARADVTLGGGAKTFAETATAGEWQGKTLREQAQARGYQ | |
| | LVSDAASLNSVTEANQQKPLLGLFADGNMPVRWLGPKATYHGNIDKP | |
| | AVTCTPNPQRNDSVPTLAQMTDKAIELLSKNEKGFFLQVEGASIDKQ | |
| | DHAANPCGQIGETVDLDEAVQRALEFAKKEGNTLVIVTADHAHASQI | |
| | VAPDTKAPGLTQALNTKDGAVMVMSYGNSEEDSQEHTGSQLRIAAYG | |
| | PHAANVVGLTDQTDLFYTMKAALGLK | |
| Alkaline | MQGACVLLLLGLWLQLSLAFIPVEEEDPAFWNRQAAQALDVAKKLQP | 5 |
| Phosphatase | IQKAAKNVILFLGDGMGVPTVTATRILKGQMNDKLGPETPLAMDQFP | |
| (B. taurus) | YVALSKTYNVDRQVPDSAGTATAYLCGVKGNYRTIGVSAAARYNQCN | |
| | TTRGNEVTSVMNRAKKAGKSVGVVTTTRVQHASPAGAYAHTVNRDWY | |
| | SDADLPADAQTYGCQDIATQLVNNMDIDVILGGGRKYMFPEGTPDPE | |
| | YPHDASVNGVRKDKRNLVQEWQAKHQGAQYVWNRTELLQAANDSSVT | |
| | HLMGLFEPADMKYNVQQDPTKDPTLEEMTEAALQVLSRNPQGFYLFV | |
| | EGGRIDHGHHDSKAYMALTEAVMFDNAIAKANELTSELDTLILVTAD | |
| | HSHVFSFGGYTLRGTSIFGLAPSKASDKKSYTSILYGNGPGYVLGGG | |
| | SRPDVNDSISEDPSYRQQAAVPLSSETHGGEDVAVFARGPQAHLVHG | |
| | VQEETFVAHVMAFAGCVEPYTDCNLPAPSGLSDAAHLAASAPSLALL | |
| | AGAMLLLLAPALY | |
| Alkaline | KAYWNKDAQDALDKQLGIKLREKQAKNVIFFLGDGMSLSTVTAARIY | 6 |
| Phosphatase | KGGLTGKFEREKISWEEFDFAALSKTYNTDKQVTDSAASATAYLTGV | |
| (P. borealis) | KTNQGVIGLDANTVRTNCSYQLDESLFTYSIAHWFQEAGRSTGVVTS | |
| | TRVTHATPAGTYAHVADRDWENDSDVVHDREDPEICDDIAEQLVFRE | |
| | PGKNFKVIMGGGRRGFFPEEALDIEDGIPGEREDGKHLITDWLDDKA | |
| | SQGATASYVWNRDDLLAVDIRNTDYLMGLFSYTHLDTVLTRDAEMDP | |
| | TLPEMTKVAIEMLTKDENGFFLLVEGGRIDHMHHANQIRQSLAETLD | |
| | MEEAVSMALSMTDPEETIILVTADHGHTLTITGYADRNTDILDFAGI | |
| | SDLDDRRYTILDYGSGPGYHITEDGKRYEPTEEDLKDINFRYASAAP | |
| | KHSVTHDGTDVGIWVNGPFAHLFTGVYEENYIPHALAYAACVGTGRT | |
| | FCDEK | |
| Intestinal- | VIPAEEENPAFWNRQAAEALDAAKKLQPIQKVAKNLILFLGDGLGVP | 7 |
| type Alkaline | TVTATRILKGQKNGKLGPETPLAMDRFPYLALSKTYNVDRQVPDSAA | |
| Phosphatase | TATAYLCGVKANFQTIGLSAAARFNQCNTTRGNEVISVMNRAKQAGK | |
| (H. sapiens) | SVGVVTTTRVQHASPAGTYAHTVNRNWYSDADMPASARQEGCQDIAT | |
| | QLISNMDIDVILGGGRKYMFPMGTPDPEYPADASQNGIRLDGKNLVQ | |
| | EWLAKHQGAWYVWNRTELMQASLDQSVTHLMGLFEPGDTKYEIHRDP | |
| | TLDPSLMEMTEAALRLLSRNPRGFYLFVEGGRIDHGHHEGVAYQALT | |
| | EAVMEDDAIERAGQLTSEEDTLTLVTADHSHVFSFGGYTLRGSSIFG | |
| | LAPSKAQDSKAYTSILYGNGPGYVFNSGVRPDVNESESGSPDYQQQA | |
| | AVPLSSETHGGEDVAVFARGPQAHLVHGVQEQSFVAHVMAFAACLEP | |
| | YTACDLAPPACTTD | |

### Polynucleotide Synthesis

In some embodiments, a method of synthesizing a polynucleotide comprises contacting (e.g., incubating) a polymerase-nucleotide conjugate with a nucleic acid, wherein a polymerase of the polymerase-nucleotide conjugate elongates the nucleic acid using its tethered nucleotide.

As described above, disclosed herein are methods of nucleic acid synthesis comprising the step of contacting (e.g., incubating) a conjugate reagent comprising a plurality of polymerase-nucleotide conjugates with a phosphatase. In some embodiments, contacting a conjugate reagent comprising a plurality of polymerase-nucleotide conjugates with a phosphatase occurs before or during cyclic extension reactions. In some embodiments, the presence of a phosphatase in a stepwise method of nucleic acid synthesis reduces non-terminations and processes that lead to addition of more than one nucleotide per step. In some embodiments, use of a conjugate reagent treated with (e.g., incubated with) a phosphatase reduces non-terminations when the conjugate reagent is used in a stepwise method of nucleic acid synthesis as compared to an untreated conjugate reagent.

### Timing of Addition or Removal of Phosphatases

Polymerase-nucleotide conjugates may be stored together with a phosphatase and remain in the system (also during the DNA extension reaction) or a phosphatase may be removed, for a certain incubation period before the conjugates are added to the DNA, and upon initiation of the DNA addition reaction.

In some embodiments a phosphatase is incubated with a conjugate reagent comprising a plurality of polymerase-nucleotide conjugates. In some embodiments, incubation of a conjugate reagent with a phosphatase is performed before contacting a sample with the conjugate reagent. In some embodiments, a phosphatase is removed from a conjugate reagent prior to contacting a sample with the conjugate reagent. In some embodiments, incubation of a conjugate reagent with a phosphatase is performed after contacting a sample with the conjugate reagent.

### Phosphatase Concentration

The concentration of phosphatase in contact or incubated with the polymerase-nucleotide conjugate (e.g., a conjugate reagent) can be expressed in, for example, a stoichiometric ratio of phosphatase to conjugate fold increase relative to the conjugate concentration, units of activity of phosphatase, molarity, or mg/mL.

Any suitable stoichiometric ratio of conjugate to phosphatase can be used in the methods described herein. In some embodiments, the stoichiometric ratio of conjugate to phosphatase is from about 1:1 to about 1:500. In some embodiments, the stoichiometric ratio of conjugate to phosphatase is about 1:1, about 1:2, about 1:3, about 1:4, about 1:5, about 1:10, about 1:15, about 1:20, about 1:25, about 1:30, about 1:35, about 1:40, about 1:45, about 1:50, about 1:55, about 1:60, about 1:65, about 1:70, about 1:75, about 1:80, about 1:85, about 1:90, about 1:95, about 1:100, about 1:105, about 1:110, about 1:115, about 1:120, about 1:125, about 1:130, about 1:135, about 1:140, about 1:145, about 1:150, about 1:155, about 1:160, about 1:165, about 1:170 about 1:175, about 1:180, about 1:185, about 1:190, about 1:195, about 1:200, about 1:225, about 1:250, about 1:275, about 1:300, about 1:325, about 1:350, about 1:375, about 1:400, about 1:425, about 1:450, about 1:475, or about 1:500.

Any suitable stoichiometric ratio of phosphatase to conjugate can be used in the methods described herein. In some embodiments, the stoichiometric ratio of phosphatase to conjugate is from about 1:1 to about 1:500. In some embodiments, the stoichiometric ratio of conjugate to phosphatase is about 1:1, about 1:2, about 1:3, about 1:4, about 1:5, about 1:10, about 1:15, about 1:20, about 1:25, about 1:30, about 1:35, about 1:40, about 1:45, about 1:50, about 1:55, about 1:60, about 1:65, about 1:70, about 1:75, about 1:80, about 1:85, about 1:90, about 1:95, about 1:100, about 1:105, about 1:110, about 1:115, about 1:120, about 1:125, about 1:130, about 1:135, about 1:140, about 1:145, about 1:150, about 1:155, about 1:160, about 1:165, about 1:170 about 1:175, about 1:180, about 1:185, about 1:190, about 1:195, about 1:200, about 1:225, about 1:250, about 1:275, about 1:300, about 1:325, about 1:350, about 1:375, about 1:400, about 1:425, about 1:450, about 1:475, or about 1:500.

Any suitable phosphatase concentration can be used in the methods described herein. In some embodiments, the phosphatase concentration is from about 0.01 mg/mL to about 10.5 mg/mL. In some embodiments, the phosphatase concentration is about 0.1 mg/mL, about 0.15 mg/mL, about 0.25 mg/mL, about 0.5 mg/mL, about 0.75 mg/mL, about 1 mg/mL, about 1.25 mg/mL, about 1.5 mg/mL, about 1.75 mg/mL, about 2 mg/mL, about 2.25 mg/mL, about 2.5 mg/mL, about 2.75 mg/mL, about 3, about 3.25 mg/mL, about 3.5 mg/mL, about 3.75 mg/mL, about 4 mg/mL, about 4.25 mg/mL, about 4.5 mg/mL, about 4.75 mg/mL, about 5, about 5.25 mg/mL, about 5.5 mg/mL, about 5.75 mg/mL, about 6 mg/mL, about 6.25 mg/mL, about 6.5 mg/mL, about 6.75 mg/mL, about 7, about 7.25 mg/mL, about 7.5 mg/mL, about 7.75 mg/mL, about 8 mg/mL, about 8.25 mg/mL, about 8.5 mg/mL, about 8.75 mg/mL, about 9, about 9.25 mg/mL, or about 9.5 mg/mL, about 9.75 mg/mL, about 10 mg/mL, about 10.25 mg/mL, or about 10.5 mg/mL.

Any suitable fold increase of phosphatase over conjugate can be used in the methods described herein. In some embodiments, the fold increase of phosphatase over conjugate is from about 2-fold to about 500-fold. In some embodiments, the fold increase of phosphatase over conjugate is about 2-fold, about 3-fold, about 4-fold, about 5-fold, about 10-fold, about 15-fold, about 20-fold, about 25-fold, about 30-fold, about 35-fold, about 40-fold, about 45-fold, or about 50-fold, about 55-fold, about 60-fold, about 65-fold, about 70-fold, about 75-fold, about 80-fold, about 85-fold, about 90-fold, about 95-fold, about 100-fold, about 105-fold, about 110-fold, about 115-fold, about 120-fold, about 125-fold, about 130-fold, about 135-fold, about 140-fold, about 145-fold, or about 150-fold, about 155-fold, about 160-fold, about 165-fold, about 170-fold, about 175-fold, about 180-fold, about 185-fold, about 190-fold, about 195-fold, about 200-fold, about 225-fold, about 250-fold, about 275-fold, about 300-fold, about 325-fold, or about 350-fold, about 375-fold, about 400-fold, about 425-fold, about 450-fold, about 475-fold, or about 500-fold.

Any suitable fold increase of conjugate over phosphatase can be used in the methods described herein. In some embodiments, the fold increase of conjugate over phosphatase is from about 2-fold to about 500-fold. In some embodiments, the fold increase of conjugate over phosphatase is about 2-fold, about 3-fold, about 4-fold, about 5-fold, about 10-fold, about 15-fold, about 20-fold, about 25-fold, about 30-fold, about 35-fold, about 40-fold, about 45-fold, or about 50-fold, about 55-fold, about 60-fold, about 65-fold, about 70-fold, about 75-fold, about 80-fold, about 85-fold, about 90-fold, about 95-fold, about 100-fold, about 105-fold, about 110-fold, about 115-fold, about 120-fold, about 125-fold, about 130-fold, about 135-fold, about 140-fold, about 145-fold, or about 150-fold, about 155-fold, about 160-fold, about 165-fold, about 170-fold, about 175-fold, about 180-fold, about 185-fold, about 190-fold, about 195-fold, about 200-fold, about 225-fold, about 250-fold, about 275-fold, about 300-fold, about 325-fold, or about 350-fold, about 375-fold, about 400-fold, about 425-fold, about 450-fold, about 475-fold, or about 500-fold.

Any suitable concentration of phosphatase can be used in the methods described herein. In some embodiments, the concentration of phosphatase is from about 0.5 μM to about 500 μM. In some embodiments, the concentration of phosphatase is about 0.5 μM, about 1 μM, about 2 μM, about 5 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 80 μM, about 85 μM, about 90 μM, about 95 μM, about 100 μM, about 105 μM, about 110 μM, about 115 μM, about 120 μM, about 125 μM, about 130 μM, about 135 μM, about 140 μM, about 145 μM, about 150 μM, about 155 μM, about 160 μM, about 165 μM, about 170 μM, about 180 μM, about 185 μM, about 190 μM, about 195 μM, about 200 μM, about 225 μM, about 250 μM, about 275 μM, about 300 μM, about 325 μM, about 350 μM, about 375 μM, about 400 μM, about 425 μM, about 450 μM, about 475 μM, or about 500 μM.

### Rates of Non-Termination

In some embodiments, presence of a phosphatase in a stepwise method of nucleic acid synthesis reduces non-terminations and processes that lead to addition of more than one nucleotide per step. Polynucleotides or nucleic acids generated in the methods described herein are said to contain an insertion if a non-termination event has occurred. In some embodiments, nucleic acid synthesis in the presence of a phosphatase reduces the rate of non-terminations by about 50% to about 100% compared to nucleic acid synthesis in the absence of a phosphatase. In some embodiments, rates of non-terminations are reduced by about 50%, about 55%, about 60%, about 65%, about 70%, about 75%, about 80%, about 85%, about 90%, about 95%, about 96%, about 97%, about 98%, about 99%, or about 100% compared to nucleic acid synthesis in the absence of a phosphatase. In some embodiments, the total amount of nucleic acid synthesis product with insertions generated in the presence of a phosphatase is less than about 5%, less than about 4%, less than about 3%, less than about 2%, less than about 1%, less than about 0.5%, less than about 0.1%, less than about 0.05%, or less than about 0.01%. In some embodiments, nucleic acid synthesis product generated in the presence of a phosphatase is absent of nucleic acid synthesis product with insertions.

### End Product

Disclosed herein, in some embodiments, are methods of synthesizing a polynucleotide comprising a pre-determined sequence, comprising contacting a conjugate reagent comprising a plurality of polymerase-nucleotide conjugates with a phosphatase. In some embodiments, contacting comprises incubating the conjugate reagent with the phosphatase. In some embodiments, the method generates a heterogeneous population of polynucleotide products comprising the pre-determined sequence. The heterogeneous population of polynucleotide products comprising the pre-determined sequence can be referred to as an “end product.”

In some embodiments, contacting the conjugate reagent comprising a plurality of polymerase-nucleotide conjugates with a phosphatase prevents insertion of nucleotides (i.e. non-terminations), such that these insertions are absent from the pre-determined sequence. In some embodiments, an end product comprises nucleic acids, a percentage of which comprise a target sequence and a percentage of which do not comprise a target sequence. In some embodiments, an end product comprises less than about 99%, less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 5%, less than about 4%, less than about 3%, less than about 2%, less than about 1%, less than about 0.5%, less than about 0.1%, less than about 0.05%, or less than about 0.01% of a polynucleotide comprising a sequence that is not the pre-determined sequence (that is, not the “target” sequence) as compared to a polynucleotide comprising a sequence that is a predetermined (“target”) sequence. In some embodiments, the end product is substantially absent of a polynucleotide comprising a sequence that is not the pre-determined (“target”) sequence.

### Analysis of End Product

Any suitable method known in the art can be used for analyzing the end product. The end product can be assessed by analyzing nucleic acid synthesis products any time following the initiation of an extension reaction (e.g. reaction time course). Analyses can be performed by, for example, capillary electrophoresis (CE) as previously demonstrated (Smith and Nelson. Curr Protoc Nucleic Acid Chem. Chapter 10: Unit 10.9. 2003; Durney et al. Anal Bioanal Chem. 407:6923-6938. 2015). CE can separate and report abundance of polynucleotide products with single nucleotide resolution. The relative abundance of each nucleic acid product generated by the methods of nucleic acid synthesis provided herein can be analyzed by CE. By comparing the abundance of the starting material (i.e. initial polynucleotide or oligonucleotide to which a nucleotide is being incorporated) and the extension products, it is possible to determine the extent to which the extension reaction is completed. The change over time of the starting material and extended species is indicative of the turnover rate, as described herein. This approach to determine turnover rate has been demonstrated previously (Palluk et al. Nat Biotech. 36 (7): 645-650. 2018). Alternatively, analysis of nucleic acid synthesis products can be performed using reverse-phase high-performance liquid chromatography (RP-HPLC) as described previously (Jensen and Davis. Biochemistry. 57 (12): 1821-1832. 2018).

CE and RP-HPLC may also be used to determine the purity of each species in a nucleic acid synthesis product by determining the area under the curve for peaks in the electropherograms and chromatograms for CE and RP-HPLC, respectively. Any suitable software package suitable for fitting curves to electropherograms and chromatograms and calculating area under the curve (AUC) may be used to determine the abundance of each polynucleotide product in a plurality of nucleotide products.

Any suitable polynucleotide sequencing method can be used for analysis (e.g., analysis of a sequence of a polynucleotide produce, e.g., intermediate product, e.g., end product, etc.). For example, the sequencing method can be long-read sequencing, next generation sequencing, short-read sequencing, shotgun sequencing, sanger sequencing, high throughput sequencing, sequencing by synthesis, sequencing by ligation, sequencing by hybridization, and/or sequencing by mass spectrometry. Sequencing can be suitable for identifying the pre-defined sequence in an end product. Sequencing can be suitable for determining the percentage of sequences in an end product that are not the pre-defined sequence.

Any method known in the art for analysis of size of an end product can be used to determine the purity of the end product. For example, gel electrophoresis and/or mass spectrometry can be used to determine purity of an end product.

### Conjugates

Disclosed herein are compositions comprising conjugates comprising nucleotides attached to a polymerase, wherein the purity of nucleotides shielded by a linked polymerase of the composition, as compared to total nucleotides in the composition, is greater than about 80%, about 85%, about 90%, about 95%, about 97%, about 98%, about 99%, about 99.5%, or about 99.9% shielded nucleotides. In some embodiments, the purity of nucleotides shielded by a linked polymerase is substantially free of impurities.

Provided herein are methods of nucleic acid synthesis using conjugates, wherein the conjugates comprise a polymerase and a nucleotide, and wherein the polymerase and the nucleotide are linked via a linker. In some embodiments, the linker comprises a selectively cleavable linkage. In some embodiments, the nucleotide is a modified nucleotide. A linker can attach to the base, the sugar, or a phosphate of a nucleotide (e.g., a wild-type nucleotide, a modified nucleotide, e.g., comprising one or more modifications relative to a wild-type nucleotide, etc.). In some embodiments, the nucleotide is a nucleotide analog. The polymerase moiety of a conjugate can elongate a nucleic acid by adding its linked nucleotide (i.e., the polymerase can catalyze the attachment of a nucleotide to which it is joined onto the 3′ end of the nucleic acid) and remains attached to the elongated nucleic acid via the linker until the linker is cleaved.

In some embodiments, the polymerase and the nucleotide are covalently linked and the distance between the linked atom of the nucleotide and the polymerase to which it is attached can be, for example, in the range of about 4-100 Å, about 15-40 Å or about 20-30 Å, or a distance appropriate for the position on the TT variant to which the nucleotide or nucleotide analog is tethered.

When a conjugate comprising a polymerase and a nucleotide is incubated with a nucleic acid, it preferentially elongates the nucleic acid using its tethered nucleotide (as opposed to using the nucleotide of another conjugate molecule).

### Linker-Nucleotide Attachment

In some embodiments, a linker of the present disclosure is attached the “5” position of pyrimidines or the “7” position of 7-deazapurines. In other embodiments, the linker may be attached to an exocyclic amine of a nucleobase, e.g. by N-alkylating the exocyclic amine of cytosine with a nitrobenzyl moiety as discussed below. The linker may be attached to any suitable atom of the nucleotide to form a conjugate, such as the phosphate, sugar, or base of the nucleotide, as will be apparent to those skilled in the art. In some embodiments, the linker is attached to the alpha-phosphate, sugar, or base of the nucleotide so that the polymerase remains attached to the nucleotide after addition to the 3′ end of an oligonucleotide. In some embodiments, the linker is attached to the β-phosphate, χ-phosphate, δ-phosphate, ε-phosphate, ϕ-phosphate, or γ-phosphate of a nucleotide. In some embodiments, the linker is attached to the terminal phosphate of a nucleotide.

Certain polymerases have a high tolerance for modification of certain parts of a nucleotide, e.g. modifications of the 5-position of pyrimidines and the 7-position of purines are well-tolerated by some polymerases (He and Seela, Nucleic Acids Research 30.24 (2002): 5485-5496; or Hottin et al., Chemistry. 2017 Feb. 10; 23 (9): 2109-2118). In some embodiments, the linker is attached to these positions.

In some embodiments, a polymerase-nucleotide conjugate is prepared by first synthesizing an intermediate compound comprising a linker and a nucleotide (referred to herein as a “linker-nucleotide”), and then the intermediate compound is attached to the polymerase. By way of non-limiting example, in some embodiments, nucleosides with substitutions compared to natural nucleosides, e.g. pyrimidines with 5-hydroxymethyl or 5-propargylamino substituents, or 7-deazapurines with 7-hydroxymethyl or 7-propargylamino substituents may be useful starting materials for preparing linker-nucleotides. An exemplary set of nucleosides with 5- and 7-hydroxymethyl substituents that may be useful for preparing linker-nucleotides is shown below:

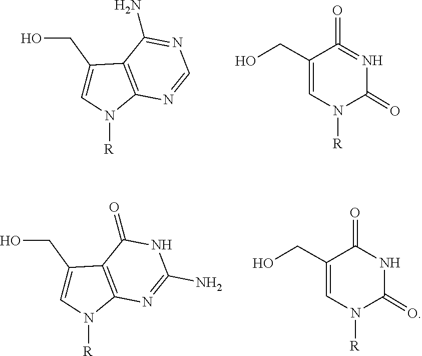

An exemplary set of nucleosides with 5- and 7-deaza-7-propargylamino substituents that may be useful for preparing linker-nucleotides is shown below:

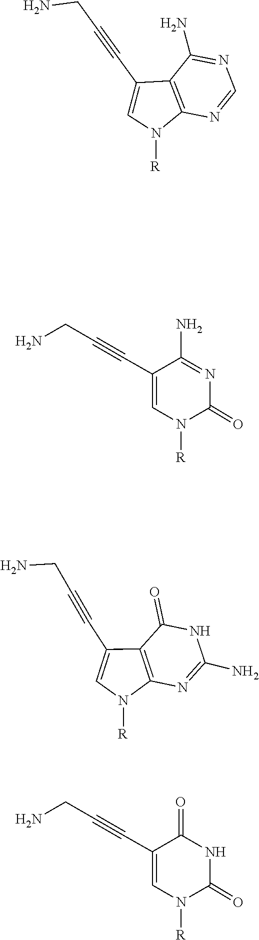

These nucleosides are also commercially available as deoxyribonucleoside triphosphates.

In some embodiments, a method of preparation (e.g., comprising an intermediate compound), the conjugate comprises a linker-nucleotide. Any suitable nucleotide may be used. In some embodiments, the linker-nucleotide comprises a nucleotide polyphosphate or a modified nucleotide polyphosphate. In some embodiments, the linker-nucleotide comprises a nucleotide triphosphate or a modified nucleotide triphosphate. In some embodiments, the linker-nucleotide comprises a nucleotide tetraphosphate or a modified nucleotide tetraphosphate. In some embodiments, the linker-nucleotide comprises a nucleotide pentaphosphate or a modified nucleotide pentaphosphate. In some embodiments, the linker-nucleotide comprises a nucleotide hexaphosphate or a modified nucleotide pentaphosphate. In some embodiments, the linker-nucleotide comprises a modified nucleobase. In some embodiments, the linker-nucleotide comprises a modified nucleobase. In some embodiments, the modified nucleobase comprises an O- or N-linked modification. In some embodiments, the O- or N-linked modification is removable following incorporation of the nucleotide portion of the linker-nucleotide into a polynucleotide. In some embodiments, the O- or N-linked modification is removable by a photolytic process. In some embodiments, the photolytic process comprises exposure to UV light, wherein the UV light comprises wavelengths at 365 nm and/or 405 nm. In some embodiments, the O- or N-linked modification is removable by a chemical process. In some embodiments, the chemical process is selected from a beta-elimination reaction, a Pd-catalyzed deallylation, and a reduction reaction. In some embodiments, the O- or N-linked modification is removable by an enzymatic process. In some embodiments, the enzymatic process comprises removal by an alkyltransferase or methyltransferase.

In some embodiments, the O- or N-linked modification reduces or eliminates Watson-Crick base pairing in a polynucleotide comprising the modified nucleobase. In some embodiments, the O- or N-linked modification reduces or eliminates secondary structure in a polynucleotide comprising the modified nucleobase. In some embodiments of the method, following removal of the O- or N-linked modification the modified nucleobase comprises a natural nucleobase. In some embodiments, the natural nucleobase is guanine, cytosine, adenine, thymine, or uracil.

### Linker-Polymerase Attachment

The conjugates provided herein comprise a polymerase tethered to a nucleotide via a linker.

Any suitable linker for tethering a nucleotide to a polymerase is contemplated for use in the methods described herein. In some embodiments, the linker is specifically attached to a cysteine residue of the polymerase using a sulfhydryl-specific attachment chemistry. Illustrative sulfhydryl specific attachment chemistries include, without limitation, ortho-pyridyl disulfide (OPSS), maleimide functionalities, 3-arylpropiolonitrile functionalities, allenamide functionalities, haloacetyl functionalities such as iodoacetyl or bromoacetyl, alkyl halides or perfluroaryl groups that can favorably react with sulfhydryls surrounded by a specific amino acid sequence (Zhang, Chi, et al. Nature chemistry 8, (2015) 120-128.). Other attachment chemistries for specific labeling of cysteine residues will be apparent to those skilled in the art or are described in the pertinent literature and texts (e.g., Kim, Younggyu, et al, Bioconjugate chemistry 19.3 (2008): 786-791.).

In some embodiments, the linker is attached to a lysine residue via an amine-reactive functionality (e.g. NHS esters, Sulfo-NHS esters, tetra- or pentafluorophenyl esters, isothiocyanates, sulfonyl chlorides, etc.). In some embodiments, the linker is attached to the polymerase via attachment to a genetically inserted unnatural amino acid, e.g. p-propargyloxyphenylalanine or p-azidophenylalanine that could undergo azide-alkyne Huisgen cycloaddition, though many suitable unnatural amino acids suitable for site-specific labeling exist and can be found in the literature (e.g. as described in Lang and Chin., Chemical reviews 114.9 (2014): 4764-4806.).

In some embodiments, the linker may be specifically attached to the polymerase N-terminus. In some embodiments, the polymerase is mutated to have an N-terminal serine or threonine residue, which may be specifically oxidized to generate an N-terminal aldehyde for subsequent coupling to e.g. a hydrazide. In some embodiments, the polymerase is mutated to have an N-terminal cysteine residue that can be specifically labeled with an aldehyde to form a thiazolidine. In some embodiments, an N-terminal cysteine residue can be labeled with a peptide linker via Native Chemical Ligation.

In some embodiments, a peptide tag sequence may be inserted into the polymerase that can be specifically labeled with a synthetic group by an enzyme, e.g. as demonstrated in the literature using biotin ligase, transglutaminase, lipoic acid ligase, bacterial sortase and phosphopantetheinyl transferase (e.g. as described in refs. 74-78 of Stephanopoulos & Francis Nat. Chem. Biol. 7, (2011) 876-884).

In some embodiments, the linker is attached to a labeling domain fused to the polymerase. For example, a linker with a corresponding reactive moiety may be used to covalently label SNAP tags, CLIP tags, HaloTags and acyl carrier protein domains (e.g. as described in refs. 79-82 of Stephanopoulos & Francis Nat. Chem. Biol. 7, (2011) 876-884).

In some embodiments, the linker is attached to an aldehyde specifically generated within the polymerase, as described in Carrico et al. (Nat. Chem. Biol. 3, (2007) 321-322). For example, after insertion of an amino acid sequence that is recognized by the enzyme formylglycine-generating enzyme (FGE) into the polymerase, it may be exposed to FGE, which will specifically convert a cysteine residue in the recognition sequence to formylglycine (i.e. producing an aldehyde). This aldehyde may then be specifically labeled with e.g. a hydrazide or aminooxy moiety of a linker.

In some embodiments, a linker may be attached to the polymerase via non-covalent binding of a moiety of the linker to a moiety fused to the polymerase. Examples of such attachment strategies include fusing a polymerase to streptavidin that can bind a biotin moiety of a linker, or fusing a polymerase to anti-digoxigenin that can bind a digoxigenin moiety of a linker. In some embodiments, site-specific labeling may lead to an attachment of the linker to the polymerase that may readily be reversed (e.g. an ortho-pyridyl disulfide (OPSS) group that forms a disulfide bond with a cysteine that can be cleaved using reducing agents, e.g. using TCEP), other attachment chemistries will produce permanent attachments.

In some embodiments, the polymerase is mutated to ensure specific attachment of the tethered nucleotide to a particular location of the polymerase, as will be apparent to those skilled in the art. For example, with sulfhydryl-specific attachment chemistries such as maleimides or ortho-pyridyl disulfides, accessible cysteine residues in the wild-type polymerase may be mutated to a non-cysteine residue to prevent labeling at those positions. On this “reactive cysteine-free” background, a cysteine residue may be introduced by mutation at the desired attachment position. These mutations preferentially do not interfere with the activity of the polymerase.

Other strategies for site-specific attachment of synthetic groups to proteins will be apparent to those skilled in the art and are reviewed in literature, (e.g. Stephanopoulos & Francis Nat. Chem. Biol. 7, (2011) 876-884).

### Shielding

As described herein, in some embodiments, a polymerase (e.g., template-independent polymerase) remains attached to a nucleic acid via a tether to an added nucleotide until exposed to some stimulus that causes cleavage of the linkage to the added nucleotide. In this situation, further extensions by polymerase-nucleotide conjugates are hindered (i.e., the nucleotide is “shielded”) when: 1) the attached polymerase molecule hinders other conjugates from accessing the 3′ OH of the extended DNA molecule and 2), other nucleotides in the system are hindered from accessing the catalytic site of the polymerase that remains attached to the 3′ end of the extended nucleic acid. (The extent of shielding may be described as the extent to which both of these interactions are hindered.) To enable subsequent extensions, the linker tethering the incorporated nucleotide to the polymerase can be cleaved, releasing the polymerase from the nucleic acid and therefore re-exposing its 3′ OH group for subsequent elongation.

In some embodiments employing shielding, the linker can be attached to any atom in the nucleobase, sugar, or α-phosphate, as will be apparent to those skilled in the art.

Methods for nucleic acid synthesis provided herein that employ the shielding effect to achieve termination comprise an extension step wherein a nucleic acid is exposed to conjugates preferentially in the absence of free (i.e., untethered) nucleoside triphosphates, because the termination mechanism of shielding may not prevent their incorporation into the nucleic acid.

In some embodiments, termination of further elongation may be “complete”, meaning that after a nucleic acid molecule has been elongated by a conjugate, further elongations cannot occur during the reaction. In other embodiments, termination of further elongation may be “incomplete”, meaning that further elongations can occur during the reaction but at a substantially decreased rate compared to the initial elongation, e.g., 100 times slower, or 1000 times slower, or 10,000 times slower, or more. Conjugates that achieve incomplete termination may still be used to extend a nucleic acid by predominantly a single nucleotide (e.g., in methods for nucleic acid synthesis and sequencing) when the reaction is stopped after an appropriate amount of time. In some embodiments, the reagent containing the conjugate may additionally contain polymerases without tethered nucleotides, but those polymerases should not significantly affect the reaction because there are no free dNTPs in the mix.

Reagents based on conjugates employing the shielding effect to achieve termination preferentially only contain polymerase-nucleotide conjugates in which all polymerases remain folded in the active conformation. In some cases, if the polymerase moiety of a conjugate is unfolded, its tethered nucleotide may become more accessible to the polymerase moieties of other conjugate molecules. In these cases, the unshielded nucleotides may be more readily incorporated by other conjugate molecules, circumventing the termination mechanism.

Polymerase-nucleotide conjugates employing the shielding effect to achieve termination are preferentially only labeled with a single nucleotide moiety. Polymerase-nucleotide conjugates labeled with multiple nucleotides that can access the catalytic site can, in some cases, incorporate multiple nucleotides into the same nucleic acid. Additional tethered nucleotides may therefore lead to additional, undesired nucleotide incorporations into a nucleic acid during a reaction. Furthermore, only one tethered nucleotide can occupy the (buried) catalytic site of its polymerase at a time so the other tethered nucleotide(s) may have an increasing accessibility to the polymerase moieties of other conjugate molecules, as discussed below.

Polymerase-nucleotide conjugates employing the shielding effect to achieve termination preferentially comprise as short of a linker as possible that still enables the nucleotide to frequently access the catalytic site of its tethered polymerase molecule in a productive conformation, in order to enable fast incorporation of the nucleotide into a nucleic acid. Such conjugates may also preferentially employ an attachment position of the linker to the polymerase as close to the catalytic site as possible, enabling use of a shorter linker. The length of the linker will determine the maximum distance from the attachment point a tethered nucleotide or a tethered nucleic acid can reach. A smaller distance may lead to a reduced accessibility of the tethered moiety to other polymerase-nucleotide molecules, as discussed below. In some embodiments, linkers are approximately 24 and 28 Å long. Shorter linkers, e.g. with lengths of 8-15 Å, may increase shielding; while longer linkers, e.g. linkers longer than 50 Å, 70 Å or 100 Å, may reduce shielding. The shielding effect may be influenced by a combination of factors including, but not limited to, to the structure of the polymerase, the length of the linker, the structure of the linker, the attachment position of the linker to the polymerase, the binding affinity of the nucleotide to the catalytic site of the polymerase, the binding affinity of the nucleic acid to the polymerase, the preferred conformation of the polymerase, and the preferred conformation of the linker.

One contribution to shielding can be steric effects that block the 3′ OH of a nucleic acid that has been elongated by a conjugate from reaching into the catalytic site of another conjugate's polymerase moiety. Steric effects may also hinder a tethered nucleotide from reaching into the catalytic site of another polymerase-nucleotide conjugate molecule due to clashes between the conjugates that would occur during such approaches. These steric effects may result in complete termination if they completely block productive interactions between the tethered nucleotide (or elongated nucleic acid) of one conjugate molecule with another conjugate molecule, or may result in incomplete termination if they only hinder such intermolecular interactions.

Another contribution to shielding arises from the binding affinity of a tethered nucleotide to the catalytic site of a polymerase. A tethered nucleotide of a conjugate will have a high effective concentration with respect to the catalytic site of its tethered polymerase so it may remain bound to that site much of the time. When the nucleotide is bound to the catalytic site of its tethered polymerase molecule, it is unavailable for incorporation by other polymerase molecules. Thus, tethering reduces the effective concentration of nucleotides available for intermolecular incorporation (i.e. incorporation catalyzed by a polymerase molecule to which the nucleotide is not tethered). This shielding effect can enhance termination by reducing the rate by which a nucleic acid is elongated using the nucleotide moiety of one conjugate molecule by the polymerase moiety of another conjugate molecule.

Another contribution to shielding arises from the binding affinity of the 3′ region of a nucleic acid molecule to the catalytic site of a polymerase molecule. After elongation by a conjugate, the nucleic acid is tethered to the conjugate via its 3′ terminal nucleotide and will have a high effective concentration with respect to the catalytic site of its tethered polymerase so it may remain bound to that site much of the time. When the nucleic acid is bound to the catalytic site of its tethered polymerase molecule it is unavailable for elongation by other conjugate molecules. This effect can enhance termination by reducing the rate by which a nucleic acid that has been elongated by a first conjugate is further elongated by other conjugate molecules.

In some embodiments, the polymerase-nucleotide conjugates comprise additional moieties that sterically hinder the tethered nucleotide (or a tethered nucleic acid post-elongation) from approaching the catalytic sites of another conjugate molecule. Such moieties include polypeptides or protein domains that can be inserted into a loop of the polymerase, and those and other bulky molecules such as polymers that can be site-specifically ligated e.g. to an inserted unnatural amino acid or a specific polypeptide tag.

### Polymerases

As described above, when a conjugate comprising a polymerase (e.g., a template-independent polymerase) and a nucleotide is incubated with a nucleic acid or a polynucleotide, it preferentially elongates (i.e. extends) the nucleic acid or polynucleotide by incorporating the tethered nucleotide or modified nucleotide (as opposed to using the nucleotide or modified nucleotide of another conjugate molecule) into the nucleic acid or polynucleotide. In some embodiments, a polymerase in a polymerase-nucleotide conjugate is folded in an active conformation. In other embodiments, a polymerase in a polymerase-nucleotide conjugate is unfolded.

Any polymerase capable of extending a polynucleotide, incorporating a nucleotide into a polynucleotide, or incorporating a nucleotide analog into a polynucleotide is envisaged for use in the methods described herein. In some embodiments, the polynucleotide is single stranded. In some embodiments, the polynucleotide is double stranded. In some embodiments, the polynucleotide is immobilized on a solid support.

For DNA synthesis applications, in particular template-independent polymerases, e.g., a terminal deoxynucleotidyl transferase (TdT) or DNA nucleotidylexotransferase, which terms are used interchangeably to refer to an enzyme having activity as described for E.C. class 2.7.7.31 may be used.

In some embodiments methods of the present disclosure use conjugates comprising template-independent polymerases. In some embodiments, conjugates comprise a Pol-X family polymerase. In some embodiments, conjugates comprise a polymerase Terminal deoxynucleotidyl Transferase (TdT), or a variant thereof (e.g., a non-wild-type TdT, e.g., a modified TdT). In some embodiments, the template-independent polymerase is a TdT or a variant thereof (i.e., a modified TdT). In some embodiments, the TdT or variant thereof comprises a sequence sharing at least 60%, at least 65%, at least 70%, at least 75%, at least 80%, at least 85%, at least 90%, at least 95%, at least 96%, at least 97%, at least 98%, or at least 99% identity to SEQ ID NO: 1. In some embodiments of the method, the TdT comprises a sequence identical to SEQ ID NO: 1 or a portion thereof. For example, in some embodiments, the TdT comprises a sequence identical to a portion of a particular TdT (e.g., that of SEQ ID NO: 1, e.g., that of SEQ ID NO: 1, etc.). For instance, in some embodiments a given TdT may be truncated relative to the length of a particular TdT such as that set forth in SEQ ID NO: 1. In some embodiments, a TdT may be a circular permutation of SEQ ID NO: 1). In some embodiments, a TdT variant comprises one or more amino acid substitutions, insertions, deletions, and/or is a circular permutant thereof relative to a reference TdT (e.g., a wild-type TdT, a modified TdT, etc.).

In some embodiments of the method, the polymerase is a fusion protein. In some embodiments of the method, the fusion protein comprises maltose binding protein (MBP).

In some embodiments of the method, the TdT or variant thereof may be operably linked to a linker moiety including a covalent or non-covalent bond; amino acid tag (e.g., poly-amino acid tag, poly-His tag, 6His-tag (SEQ ID NO: 8)); chemical compound (e.g., polyethylene glycol); protein-protein binding pair (e.g., biotin-avidin); affinity coupling; capture probes; or any combination of these. The linker moiety can be separate from or part of a TdT variant.

>Terminal deoxynucleotidyl transferase (TdT)
(SEQ ID NO: 1)
MGGRDIVDGSEFSPSPVPGSQNVPAPAVKKISQYACORRTTLNNYNQLF
TDALDILAENDELRENEGSALAFMRASSVLKSLPFPITSMKDTEGIPSL
GDKVKSIIEGIIEDGESSEAKAVLNDERYKSFKLFTSVFGVGLKTAEKW
FRMGFRTLSKIQSDKSLRFTQMQKAGFLYYEDLVSCVNRPEAEAVSMLV
KEAVVTFLPDALVTMTGGFRRGKMTGHDVDFLITSPEATEDEEQQLLHK
VTDFWKQQGLLLYADILESTFEKFKQPSRKVDALDHFQKCFLILKLDHG
RVHSEKSGQQEGKGWKAIRVDLVMSPYDRRAFALLGWTGSRQFERDLRR
YATHERKMMLDNHALYDRTKRVFLEAESEEEIFAHLGLDYIEPWERNA

Illustrative examples of polymerases with the ability to extend single stranded nucleic acids include, but are not limited to, Polymerase Theta (Kent et al., eLife 5 (2016): el3740.), polymerase mu (Juarez et al., Nucleic acids research 34.16 (2006): 4572-4582.; or McElhinny et all, Molecular cell 19.3 (2005): 357-366.) or polymerases where template-independent activity is induced, e.g. by the insertion of elements of a template-independent polymerase (Juarez et al., Nucleic acids research 34.16 (2006): 4572-4582). In other DNA synthesis applications, the polymerase can be a template-dependent polymerase i.e., a DNA-directed DNA polymerase (which terms are used interchangeably to refer to an enzyme having activity 2.7.7.7 using the IUBMB nomenclature).

In some embodiments, such as RNA synthesis applications, tethered ribonucleotides may be used. In some such embodiments, an RNA specific nucleotidyl transferase, such as E. coli Poly(A) Polymerase (IUBMB EC 2.7.7.19) or Poly(U) Polymerase, among others, may be employed. The RNA nucleotidyl transferases can contain modifications, e.g., single point mutations, that influence the substrate specificity towards a specific rNTP (Lunde et al., Nucleic acids research 40.19 (2012): 9815-9824.). In some embodiments, a very short tether between an RNA nucleotidyl transferase and a ribonucleotide may be used to induce a high effective concentration of the nucleotide, thereby forcing incorporation of an rNTP that might not be the natural substrate of the nucleotidyl transferase.

### Linker

In some embodiments, in a conjugate of the present disclosure comprising a linker, the linker comprises atoms that connect the nucleotide to the polymerase. The linker can attach to the base, the sugar, or the α-phosphate of the nucleotide or modified nucleotide to the polymerase. In some embodiments, the polymerase and the nucleotide are covalently linked and the distance between the linked atom of the nucleotide and the polymerase to which it is attached can be, for example, in the range of about 4-100 Å, about 15-40 Å or about 20-30 Å, or a distance appropriate for the position on the polymerase to which the nucleotide is tethered. Any suitable linker for tethering the nucleotide or modified nucleotide to the polymerase is contemplated in the methods described herein. In some embodiments, the linker comprises a polyether or a polyethylene glycol (PEG). In some embodiments, the linker comprises one or more peptide bonds. In some embodiments, the linker comprises one or more sarcosines. In some embodiments, the linker comprises one or more glycines. In some embodiments, the linker comprises one or more prolines. In some embodiments, the linker comprises a carbamate. In some embodiments, the linker joins to the nucleotide at an atom of the nucleobase that is not involved in base pairing. In such embodiments, the linker is considered to be at least the atoms that connect the polymerase to any atom in the monocyclic or polycyclic ring system bonded to the Γ position of the sugar (e.g. pyrimidine or purine or 7-deazapurine or 8-aza-7-deazapurine). In some embodiments, the linker joins to the nucleotide at an atom of the nucleobase that is involved in base pairing. In some embodiments, the linker is joined to the sugar or to the α-phosphate of the nucleotide. In some embodiments, the linker is sufficiently long to allow the nucleotide to access the active site of the polymerase to which it is tethered. As described in greater detail herein, the polymerase of a conjugate is capable of catalyzing the addition of the nucleotide to which it is linked onto the 3′ end of a nucleic acid.

### Cleavage of Linker

As described herein, a linker may be attached to various positions on a nucleotide (e.g., of a conjugate of the present disclosure), and a variety of cleavage strategies may be used. It is understood that the cleavage strategy will be determined by the type of linker joining the nucleotide or modified nucleotide and the polymerase. Any suitable method for cleaving a linker is contemplated in the methods described herein.

In some embodiments, the linker is cleaved, wherein following cleavage of the linker, a nucleotide comprising a chemical group from the retained portion of the linker (i.e. a scar) is formed. Illustrative, non-limiting, chemical groups (i.e. scars) following linker cleavage are shown below. In some embodiments, the chemical group is removed by a chemical, photolytic, or enzymatic process.

In some embodiments, the linker may be cleaved by exposure to any suitable reducing agent such as dithiothreitol (DTT), β-mercaptoethanol, or tris(2-carboxyethyl)phosphine (TCEP). For example, a linker comprising a 4-(disulfaneyl)butanoyloxy-methyl group attached to the 5 position of a pyrimidine or the 7 position of a 7-deazapurine may be cleaved by reducing agents (e.g. DTT) to produce a 4-mercaptobutanoyloxymethyl scar on the nucleobase. This scar may undergo intramolecular thiolactonization to eliminate a 2-oxothiolane, leaving a smaller hydroxymethyl scar on the nucleobase. An example of such a linker attached to the 5 position of cytosine is depicted below, but the strategy is applicable to any suitable nucleobase:

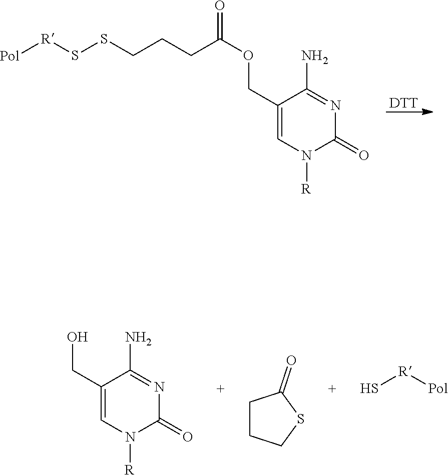

In other embodiments, the linker may be cleaved by exposure to light. For example a linker comprising a (2-nitrobenzyl)oxymethyl group may be cleaved with 365 nm light, leaving a hydroxymethyl scar, e.g. as depicted for cytosine below, but the strategy is applicable to any suitable nucleobase:

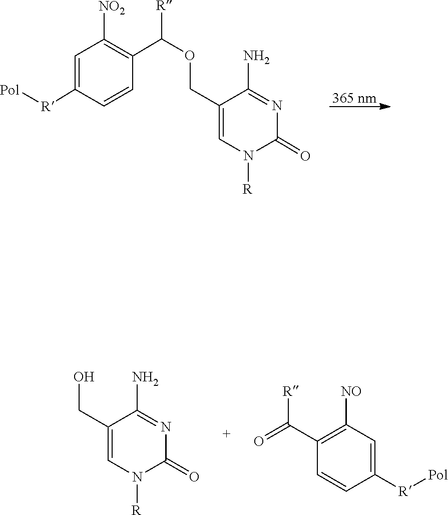

(where, e.g., R″═H or R″═CH3 or R′=i-Bu.)

In other embodiments, the linker may comprise a 3-(((2-nitrobenzyl)oxy) carbonyl) aminopropynyl group that may be cleaved with 365 nm light to release a nucleobase with a propargylamino scar. This strategy is applicable to any suitable nucleobase:

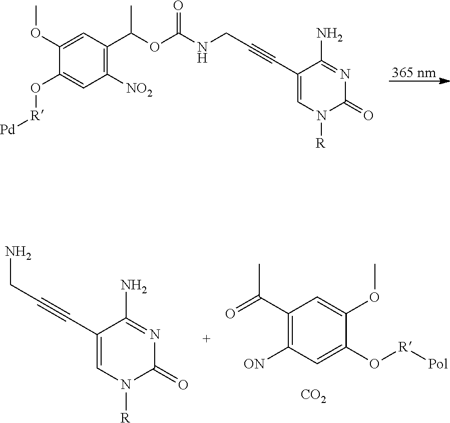

In other embodiments, the linker may comprise an acyloxymethyl group that may be cleaved with a suitable esterase to release a nucleobase with a hydroxymethyl scar, e.g. as depicted for cytosine below, but the strategy is applicable to any suitable nucleobase:

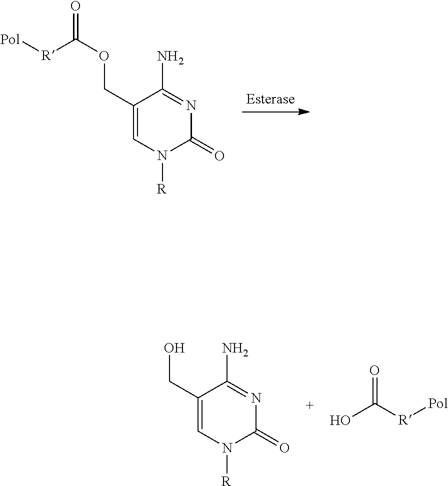

In such embodiments, the linker may comprise additional atoms (included in R′ above) adjacent to the ester that increase the activity of the esterase towards the ester bond.

In other embodiments, the linker may comprise an N-acyl-aminopropynyl group that may be cleaved with a peptidase to release a nucleobase with propargylamino scar, e.g. as depicted for 5-propargylamino cytosine below, but the strategy is applicable to any suitable nucleobase:

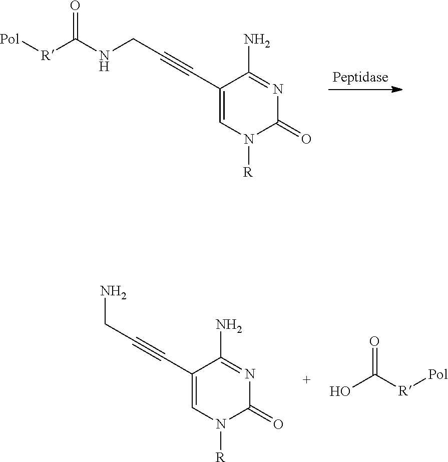

In such embodiments, the linker may comprise additional atoms (included in R′ above) adjacent to the amide that increase the activity of the peptidase towards the amide bond.

### EQUIVALENTS AND SCOPE

Those skilled in the art will recognize, or be able to ascertain using no more than routine experimentation, many equivalents to the specific embodiments in accordance with the technologies provided herein. The scope of the present disclosure is not intended to be limited to the disclosure herein, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

It is also noted that the term “comprising” is intended to be open and permits, but does not require, the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

The term “about” as used herein refers to the normal range of error for each value readily known to those skilled in the art. The term “about” value or reference to a parameter herein includes (and describes) an implementation of the value or parameter itself. For example, a description referring to “about X” includes a description of “X”. In some embodiments, “about” means a value of at most +/−10% of the recited value, e.g., +/−1%, +/−2%, +/−3%, +/−4%, +/−5%, +/−6% %, ±8%, ±9%, or ±10%.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

Below are examples of specific embodiments for carrying out technologies provided by the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of what is disclosed herein will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3rd Ed. (Plenum Press) Vols A and B (1992).

### Example 1: Preparation of Polymerase-Nucleotide Conjugates

### 1. Generation of Polymerase (TdT)

An inducible plasmid expressing murine TdT with a single cysteine located at position 182 was produced (see Palluk et al., Nature Biotechnology, 2018 for complete protocol). See also US Patent Publication No. 2019/0112627, “Nucleic Acid Synthesis and Sequencing Using Tethered Nucleoside Triphosphates” for further details of polymerase-nucleotide conjugate preparation, incorporated by reference herein in its entirety.

### 2. Protein Expression and Purification of the Polymerase

TdT expression was performed using BL21 (DE3) Gold cells (Agilent) in TB media containing antibiotics for resistance marker of the plasmid. An overnight culture of 50 mL was used to inoculate a 400 mL expression culture with 1/20 vol. Cells were grown at 37° C. and 200 rpm shaking until they reached OD 0.6. IPTG was added to a final concentration of 0.5 mM and the expression was performed for 16-20 h at 16° C. Cells were harvested by centrifugation at 8000 G for 10 min and resuspended in 20 mL buffer A (20 mM Tris-HCl, 0.5 M NaCl, pH 8)+5 mM imidazole. Cell lysis was performed using sonication followed by centrifugation at 30,000 G for 20 min. The supernatant was applied to a gravity column containing 1 mL of Ni-NTA agarose (Qiagen). The column was washed with 20 volumes of buffer A+40 mM imidazole, and bound protein was eluted using 4 mL buffer A+500 mM imidazole. The protein was concentrated to about 0.15 mL with Vivaspin 20 columns (MWCO 10 kDa, Sartorius) and then dialyzed against 200 mL TdT storage buffer (100 mM NaCl, 200 mM K2HPO4, pH 6.5) overnight using Pur-A-Lyzer™ Dialysis Kit Mini 12000 tubes (Sigma).

Ni-purified sample was applied to a HiTrap Q HP anion column. Protein was eluted with linear gradient from 100% Q Buffer A (100 mM NaCl, 20 mM K2HPO4, pH 6.5) to 100% Q Buffer B (1M NaCl, 20 mM K2HPO4, pH 6.5). SDS-PAGE analysis was used to identify fractions that contained TdT, these samples were pooled and concentrated.

### 3. Attachment of Tethered Nucleotides to the Polymerase

To prepare TdT-nucleotide conjugates, a cleavable linker-nucleotide with a moiety capable of site specifically conjugating to a cysteine (i.e., maleimide) was first synthesized. Exemplary linker-nucleotides are described in US Patent Publication No. 2019/0112627, “Nucleic Acid Synthesis and Sequencing Using Tethered Nucleoside Triphosphates.” Then, equal moles of TdT and linker-nucleotide were incubated overnight at 4° C. in 500 mM NaCl, 20 mM K₂HPO₄, at pH 6.5. TdT conjugates were separated from unreacted linker-nucleotide using a S200 size exclusion column (Cytiva) pre-equilibrated in 20 mM Tris Acetate, 50 mM Potassium Acetate; pH 7.9. Resulting conjugates comprise a conjugate comprising at least one TdT-nucleotides attached (or tethered) to a polymerase.

### Example 2: Phosphatase Prevents Unwanted Insertions During Conjugate-Based Polynucleotide Synthesis without Impacting Synthesis Kinetics

As described herein, the polymerase-nucleotide conjugates can be used to incorporate a single nucleotide of a given conjugate onto the free 3′ end of an oligonucleotide, while the polymerase can remain attached after incorporation including to prevent subsequent nucleotide addition in a controlled manner. However, insertions of more than one nucleotide can occur when using a conjugate solution for polynucleotide synthesis, negatively impacting conjugate-based polynucleotide synthesis accuracy. To test whether phosphatases can improve synthesis accuracy by preventing insertions during conjugate-based polynucleotide synthesis, we performed a single nucleotide incorporation reaction on the 3′ end of a free oligonucleotide using an A, T, C, or G nucleotide polymerase conjugate with and without phosphatase as follows:

A solution of 1 μM TdT conjugated to A, T, C, or G linker-nucleotide as prepared in Example 1 was incubated in Tris or HEPES buffer at pH 8 with a divalent metal (e.g., magnesium or cobalt), 50 mM salt (e.g., potassium acetate or NaCl), 50 nM of one of two starter DNA oligos (For A, 5′-6-FAM-T35-3′ (SEQ ID NO: 9) and for T, C, and G 5′-6-FAM-T41GCGGCGCGTTTCGCGCCGC-3′ (SEQ ID NO: 10) was used), in the presence or absence of 2 μM calf alkaline intestinal phosphatase purchased from NEB. The phosphatase was previously buffer exchanged into solution containing buffer agent at pH 8 (e.g., Tris or HEPES buffer) and salt (e.g., 50 mM NaCl or potassium acetate). Reactions were incubated at 24° C. until the reactions were stopped at 6.6 seconds; 11.6 seconds; 19.2 seconds; 65 seconds; 3 minutes 21 seconds; 9 minutes 52 seconds; and 21 minutes 41 seconds by the addition of 40 mM EDTA.

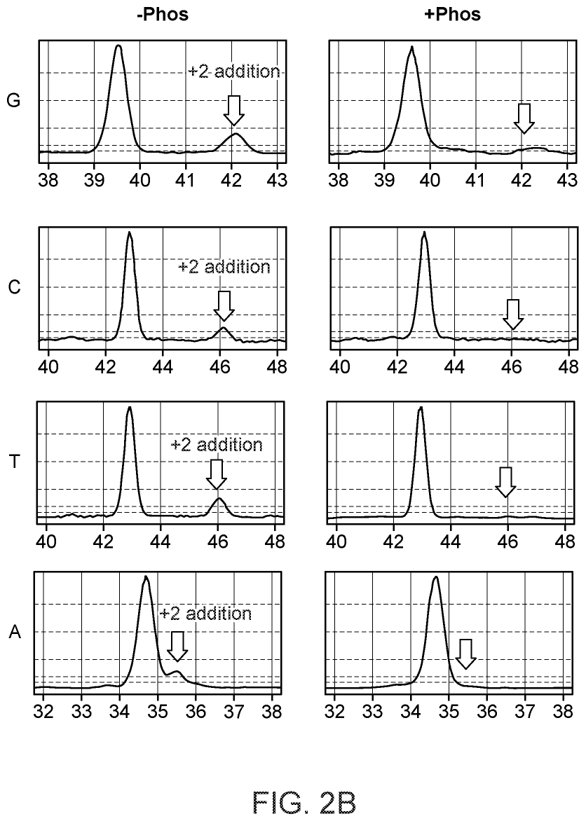

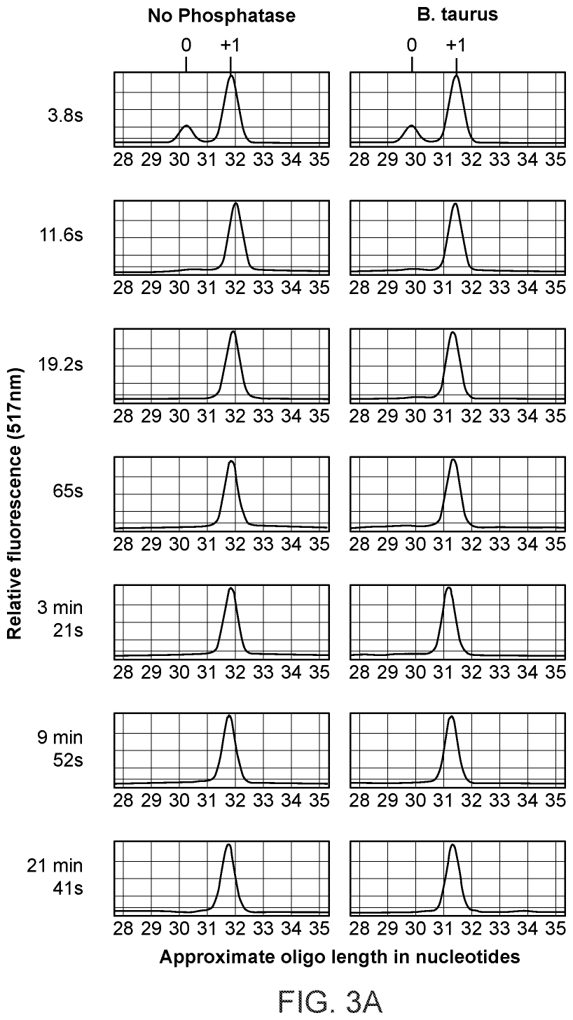

After completion of the reaction, the linker connecting the nucleotide and TdT in the conjugate was cleaved using a reagent such as a cleavage enzyme or a reducing agent to remove TdT from the oligonucleotide. Oligonucleotides were then analyzed by capillary electrophoresis to distinguish reaction products by length. Specifically, the resulting oligonucleotide addition reaction products were sized by detecting the fluorescence of the 6-FAM fluorescein bound to the starter oligo. Results are shown in FIG. 2A. The results show a first peak (starter oligo), a second peak (starter oligo with a single nucleotide added, i.e., “+1 addition”), and a third peak that emerges at later timepoints (starter oligo with two nucleotides added, i.e., “+2 addition.” That is, during the initial timepoints, the proportion of extended +1 oligo (single nucleotide incorporation, right peak) increases relative to the starter oligo (left peak), until reaching completion.

As shown in the later time points, with the final 21 min 41 sec time point shown again in FIG. 2B, another peak due to addition of 2 nucleotides to the starter oligo (+2) starts to emerge in the absence of phosphatase. The +2 addition peak is labeled in FIG. 2B and corresponds to an unwanted insertion. However, when the reaction is performed in the presence of phosphatase, the appearance of +2 additions is significantly reduced, while the rate of +1 addition is not significantly affected. Thus, synthesis using a conjugate reagent treated with phosphatase (e.g., synthesis in the presence of phosphatase) improves the accuracy of single nucleotide additions of A, C, T, and G nucleotides to a substrate by a polymerase-nucleotide conjugate and inhibits unwanted insertions.

### Example 3: Several Phosphatase Variants Prevent Unwanted Insertions During Conjugate-Based Polynucleotide Synthesis without Impacting Synthesis Kinetics

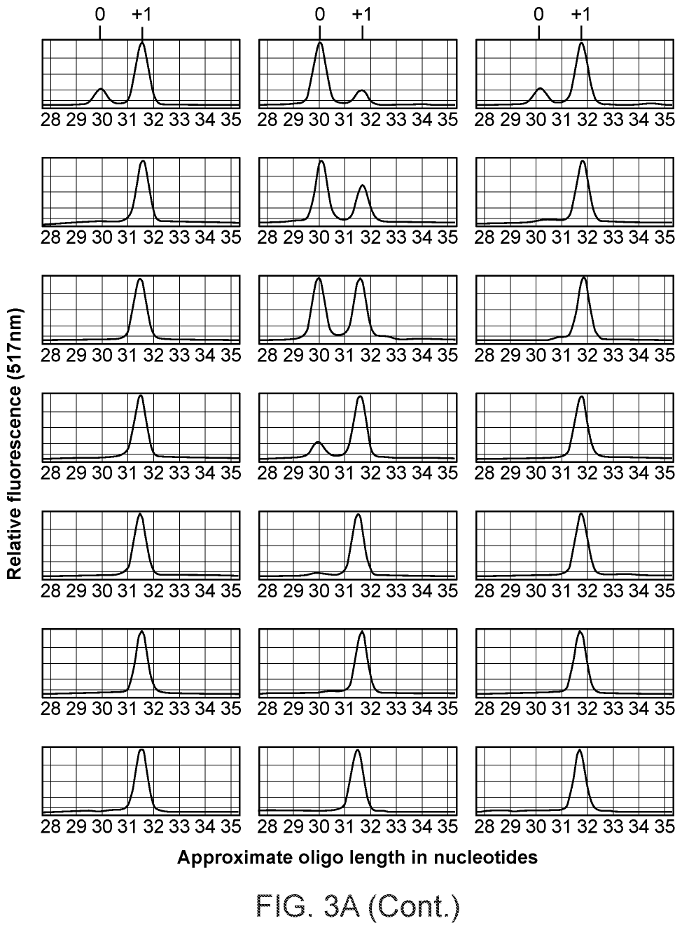

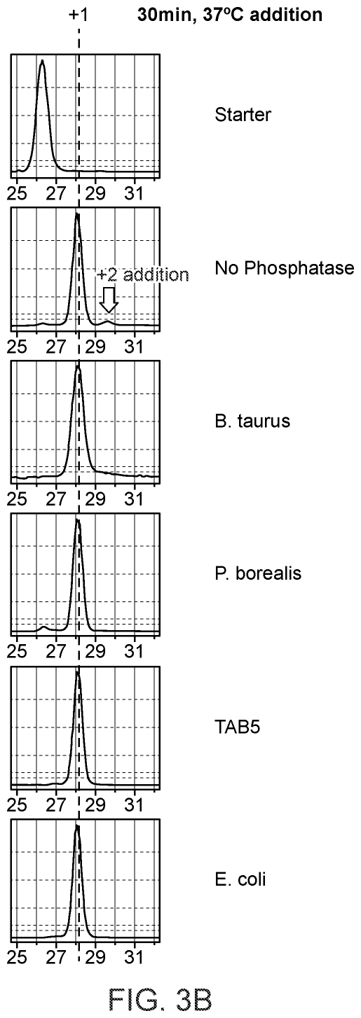

A solution of 2 μM TdT conjugated to T linker-nucleotide as prepared in Example 1 was incubated in Tris or HEPES buffer at pH 8 with a divalent metal (e.g., magnesium or cobalt), 50 mM salt (e.g., potassium acetate or NaCl), 2 μM phosphatase (or no phosphatase as a control), and 50 nM DNA oligo (5′-6-FAM-T32CCC-3′) (SEQ ID NO: 11) at 24° C. (FIG. 3A) or 37° C. (FIG. 3B). Reactions were quenched at 3.8 seconds, 11.6 seconds, 19.2 seconds, 65 seconds, 3 minutes 21 seconds, 9 minutes 52 seconds, 21 minutes 41 seconds, and 30 minutes by the addition of 40 mM EDTA. Phosphatases were previously buffer exchanged into solutions containing buffer agent at pH 8 (e.g., Tris or HEPES) and salt (e.g., 50 mM NaCl or potassium acetate). The following phosphatase variants were tested: B. taurus (Quick CIP, NEB), P. borealis (shrimp alkaline phosphatase, NEB), Antarctic bacterium TAB5 (Antarctic phosphatase, NEB), and E. coli (Takara Bio) phosphatase.

After completion of the reaction, the linker connecting the nucleotide and TdT in the conjugate was cleaved using a reagent such as a cleavage enzyme or a reducing agent to remove TdT from the oligonucleotide. The resulting synthesized oligonucleotides in 3A were analyzed by capillary electrophoresis to distinguish oligo populations by length, with “0” assigned to represent the peak for the starter oligo and “+1” representing the peak for a starter oligo with a single incorporated T at its 3′ end; and in 3B, the results show a visible peak for oligos with 2 incorporated nucleotides “+2 addition” in the ‘No Phosphatase’ control.

Specifically, oligonucleotides were analyzed by capillary electrophoresis and using fluorescence detection of the 6-FAM fluorescein bound to the starting oligo to distinguish reaction products by length. The resulting oligonucleotide addition reaction products after variable addition times between 3.8 seconds and 21 minutes 41 seconds at 24° C. are shown in FIG. 3A. The resulting oligonucleotide addition reaction products after an addition time of 30 minutes at 37° C. are shown in FIG. 3B, with arrows designating the expected size of +2 additions.

As shown, the appearance of +2 additions is significantly decreased by all timepoints tested as compared to ‘No Phosphatase’ (FIG. 3B), while the +1 addition is completed in the presence of all phosphatases by the 21 minute 41 second timepoint (FIG. 3A). Furthermore, all but the TAB5 phosphatase variant did not visibly impact the kinetics of nucleotide incorporation as compared to ‘no phosphatase’. Thus, all phosphatase variants tested are suitable for inhibiting unwanted insertions during polynucleotide synthesis, with most variants also suitable where fast incorporation kinetics are desired.

### Example 4. Phosphatase Inhibits Unwanted Insertions During a Stepwise Controlled Synthesis of a 50-Mer Polynucleotide Using Polymerase-Nucleotide Conjugates

Cycled polynucleotide synthesis using nucleotide-polymerase conjugates was performed to create a defined 50-mer sequence:

(SEQ ID NO: 12)
5′-AACCGACCAAGCTACGGTTCAGAAAATTCGCGATGCAATTCGCGA
TCAGC-3′.

Synthesis was performed starting at the 3′ end of a FAM labeled DNA oligo (starter oligo):

5′-6-FAM-CTGACAGAGATGATGAAGTCACATGAGACATGAACTGAGTCTTTT-3′ (SEQ ID NO: 13) hybridized to a DNA that was attached to a surface.

Two 50-mer synthesis reactions were performed, one in the presence of phosphatase and the other one without phosphatase. DNA extension was performed on the starting molecule by cycled addition of nucleotides via TdT-nucleotide conjugates. Each DNA extension cycle to add one nucleotide to the 3′ end of the polynucleotide bound to the surface was performed as follows (at a temperature between 24-37° C.):

1. Nucleotide addition: A solution of TdT-nucleotide conjugate (corresponding to either A, T, C, or G), a divalent metal (e.g., cobalt or magnesium), and 50 mM salt (e.g., potassium acetate or NaCl) in Tris or HEPES buffer at pH 8, with or without 3 μM E. coli phosphatase was added to DNA bound to a surface (e.g., a starter oligo).

2. Removal of oligo-bound TdT: After incubating for sufficient time for addition of the TdT-nucleotide conjugate to the surface-bound oligo, 40 mM EDTA was added to terminate the TdT extension reaction, and the linker connecting the nucleotide and TdT in the conjugate was cleaved using a reagent such as a cleavage enzyme or a reducing agent.

3. Regeneration of the surface: A solution of NaOH pH 11 with 0.5 M NaCl was then used to wash away all unbound reaction components from the surface.

Steps 1-3 were repeated to generate the desired polynucleotide sequence.

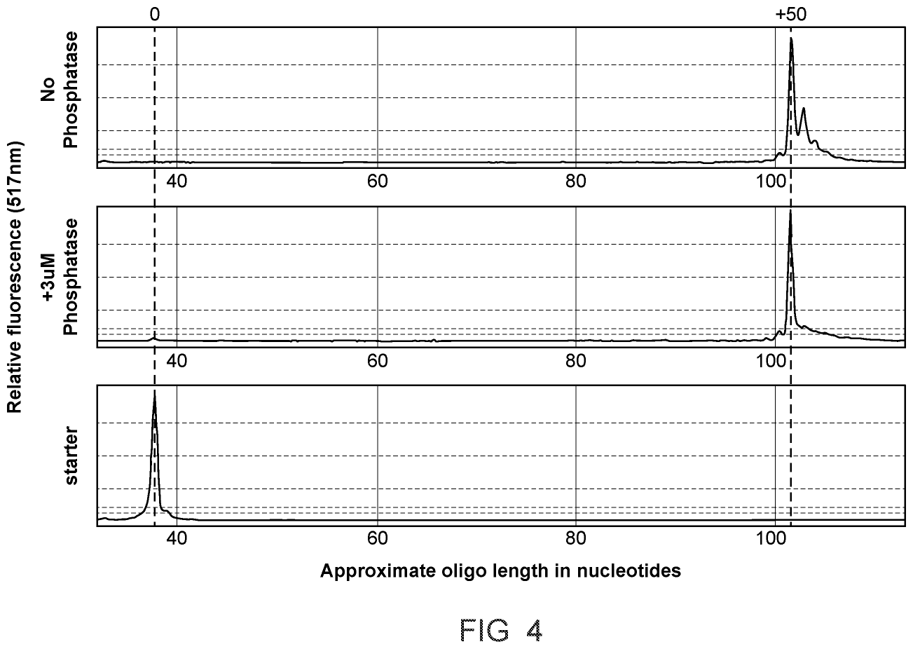

The resulting synthesized polynucleotides were removed from the surface and analyzed by detecting FAM fluorescence on a SeqStudio Genetic Analyzer (ThermoFisher) DNA Analyzer to distinguish populations of polynucleotides by length. The results are shown in FIG. 4.

As shown in FIG. 4, the synthesis without phosphatase shows products with greater than 50 bases (representing products with 1 or more non-termination events or insertions) on the order of 40% of total substrate. However, in the presence of phosphatase, synthesized oligonucleotides greater than 50 bases only make up about 10% of synthesized population, indicating a significantly reduced insertion rate at each synthesis step. Furthermore, there is no detectable change in deletions (cycles where no addition occurred) in the presence or absence of phosphatase.

The above results show that the presence of phosphatase during a conjugate-based polynucleotide synthesis leads to substantial (at least 4×) decrease in insertions with no effect on deletion rates in multi-step synthesis reactions compared to reactions without phosphatases. This represents a significant improvement in the quality of polynucleotide synthesis by polymerase-nucleotide conjugates.

### OTHER EMBODIMENTS

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A method of nucleic acid synthesis, comprising:
(i) providing a conjugate reagent comprising a plurality of polymerase-nucleotide conjugates, wherein said conjugates each comprise a nucleotide covalently attached to a polymerase via a linker; and
(ii) contacting a sample comprising a polynucleotide with said conjugate reagent, wherein said polymerase of the conjugate catalyzes the covalent addition of the nucleotide of the polymerase-nucleotide conjugate onto the 3′ hydroxyl of said polynucleotide, and
wherein said conjugate reagent is or has been incubated with a phosphatase, wherein said phosphatase is capable of removing a terminal 5′ phosphate of an unshielded nucleotide.

2. A method, comprising:
(i) providing a conjugate reagent comprising a polymerase-nucleotide conjugate; and
(ii) contacting the conjugate reagent with a phosphatase,
wherein said conjugate comprises a nucleotide covalently attached to a polymerase via a linker, and
wherein said phosphatase is capable of removing a terminal 5′ phosphate of an unshielded nucleotide.

3. The method of claim 1 or claim 2, wherein the nucleotide is a modified nucleotide.

4. The method of claim 1, further comprising cleaving said linker to remove the polymerase attached to the polynucleotide after addition of said conjugate.

5. The method of claim 1, further comprising incubating said conjugate reagent with said phosphatase and said incubating hydrolyzes at least one terminal 5′ phosphate of at least one unshielded nucleotide.

6. The method of claim 5, wherein said incubation of said conjugate reagent with said phosphatase is performed before contacting said sample with said conjugate reagent.

7. The method of claim 6, wherein said phosphatase is removed from said conjugate reagent prior to contacting said sample with said conjugate reagent.

8. The method of claim 5, wherein said incubation of said conjugate reagent with said phosphatase is performed after contacting said sample with said conjugate reagent.

9. The method of any one of claim 1 or 3-8, further comprising repeating each of the steps as recited in any one of claim 1 or 3-8 one or more times to synthesize an elongated polynucleotide.

10. The method of claim 1 or 2, wherein said phosphatase does not remove a terminal 5′ phosphate of a shielded nucleotide of said conjugate.

11. The method of claim 1 or 2, wherein said unshielded nucleotide is not attached to a polymerase.

12. The method of claim 1 or 2, wherein said unshielded nucleotide is part of a conjugate:
wherein the polymerase is unfolded or improperly folded,
wherein the nucleotide is attached to the polymerase such that the nucleotide is not shielded from the phosphatase, or wherein multiple nucleotides are attached to the polymerase.

13. The method of claim 1 or 2, wherein said phosphatase is an alkaline phosphatase or a non-alkaline phosphatase.

14. The method of claim 1 or 2, wherein said polymerase comprises a template-independent polymerase.

15. The method of claim 14, wherein said template-independent polymerase is Terminal deoxynucleotidyl Transferase (TdT), or a variant thereof.

16. The method of claim 1 or 2, wherein said polymerase comprises a template-dependent polymerase.

17. The method of claim 16, wherein said polymerase comprises a DNA polymerase.

18. The method of claim 16, wherein said polymerase comprises an RNA polymerase.

19. The method of claim 9, wherein the rate of non-termination in said polynucleotide synthesis is decreased as compared to the same synthesis using an otherwise identical conjugate reagent that has not been incubated with a phosphatase.

20. The method of claim 1 or 2, wherein the linker is a cleavable linker.

21. The method of claim 1 or 2, wherein the phosphatase removes the terminal 5′ phosphate of the unshielded nucleotide.

22. A composition comprising a plurality of conjugates, wherein each conjugate comprises a nucleotide or modified nucleotide attached to a polymerase, wherein purity of nucleotides shielded by a linked polymerase of the composition is greater than about 80%, about 85%, about 90%, about 95%, about 97%, about 98%, about 99%, about 99.5%, or about 99.9%, or about 99.9% with reference to total quantity of nucleotides in the composition.

23. The composition of claim 22, wherein said plurality of conjugates comprises less than about 5%, less than about 4%, less than about 3%, less than about 2%, less than about 1%, less than about 0.5%, less than about 0.1%, less than about 0.05%, or less than about 0.01% of unshielded nucleotides or modified nucleotides.

24. The composition of claim 22 or claim 23, further comprising a phosphatase.

25. The composition of any one of claims 22 to 24, wherein the plurality of conjugates is capable of extending a nucleic acid molecule by one nucleotide.

26. The composition of any one of claims 22 to 24, wherein the plurality of conjugates is capable of extending a nucleic acid molecule by not more than one nucleotide.

27. The composition of claim 25 or claim 26, wherein the nucleic acid molecule is single stranded.

28. The composition of claim 25 or claim 26, wherein the nucleic acid is double stranded.

29. A method of synthesizing a polynucleotide comprising a pre-determined sequence comprising contacting a nucleic acid molecule with the composition of any one of claims 22 to 26.

30. The method of claim 29, wherein the method generates a polynucleotide product comprising the pre-determined sequence.

31. The method of claim 30, wherein the polynucleotide product has less than about 5%, less than about 4%, less than about 3%, less than about 2%, less than about 1%, less than about 0.5%, less than about 0.1%, less than about 0.05%, or less than about 0.01% of a polynucleotide comprising a sequence that is not the pre-determined sequence as compared to all polynucleotides in the product.

32. The method of claim 29, wherein the nucleic acid molecule is single stranded.

33. The method of claim 29, wherein the nucleic acid molecule is double stranded.

34. A method, comprising: treating a composition comprising a polymerase-nucleotide conjugate comprising a step of contacting said composition with a phosphatase, wherein said phosphatase is capable of removing a terminal 5′ phosphate of an unshielded nucleotide.

35. A method of reducing one or more non-termination reactions in a nucleic acid molecule synthesis, wherein the synthesis is conducted in an environment comprising one or more unshielded nucleotides and wherein the reduction comprises: contacting a conjugate reagent comprising a polymerase-nucleotide conjugate with a phosphatase, wherein the polymerase-nucleotide conjugate comprises a nucleotide tethered to the polymerase by a linker, and wherein the phosphatase is capable of removing the terminal 5′ phosphate of the one or more unshielded nucleotides.

36. The method of claim 36, wherein the number of non-terminations occurring in a synthesis reaction performed in the presence of a phosphatase is reduced as compared to a synthesis conducted under the same conditions but in the absence of a phosphatase.

37. The method of any one of claims 34-37, wherein the nucleotide is a modified nucleotide.
